(19) United States
(12) Patent Application Publication
(10) Pub. No.: US 2012/0109042 A1
(43) Pub. Date: May 3, 2012

(54) Methods of Treating Diseased Tissue

(76) Inventors: John Koo, San Francisco, CA (US); Tina Bhutani, San Francisco, CA (US)

(21) Appl. No.: 13/282,284

(22) Filed: Oct. 26, 2011

Related U.S. Application Data
(60) Provisional application No. 61/407,844, filed on Oct. 28, 2010.

(51) Int. Cl. A61M 37/00

(52) U.S. Cl. 604/20

(57) ABSTRACT

Two or three external therapeutic options such as clobetasol propionate spray, calcitriol ointment, and excimer laser phototherapy are used to treat generalized psoriasis with systemic safety and better efficacy than any systemic or biologic agent. Combinations of these agents, for example used in parallel and/or series for specific durations, can achieve a significant result: a PASI 75 response after only four weeks and/or a PASI 95-100 response after only six weeks.

### RELATED APPLICATIONS

This application claims priority benefit under 35 U.S.C. §119(e) of U.S. Provisional Application Ser. No. 61/407,844, filed Oct. 28, 2010, which is hereby incorporated by reference in its entirety.

### BACKGROUND

1. Field

The present application generally relates to methods of treating skin disorders, and, more specifically, to treatment protocols for psoriasis.

2. Description of the Related Art

Skin disorders including atopic dermatitis, dyshidrosis, eczema, lichen planus, psoriasis, and vitiligo are conditions that commonly affect large populations at some time in their lives. For example, about 1% to about 3% of the population is estimated to be afflicted with psoriasis. Although the disease's prevalence in the United States is not as well understood, it appears that between 150,000 and 260,000 new cases are diagnosed each year. This suggests that at least several million people suffer from the disease in the United States.

Psoriasis can range in severity from relatively mild, with some drying and flaking of the affected skin, to severe cases with very severe outbreaks over large areas of the patient's body. Approximately one-third of patients experience moderate to severe psoriasis. Even very mild psoriasis is uncomfortable and unsightly. Severe cases can be physically and psychologically debilitating, presenting a very serious threat to the patient's overall health.

Psoriasis can be divided into various types according to the affected area and/or symptoms. For example, plaque psoriasis (e.g., psoriasis vulgaris) is a common form of the condition and accounts for about 80% to about 90% of patients. Plaque psoriasis typically appears as red patches or plaques with dry, silvery scales. Another type is guttate psoriasis, which is characterized by numerous small round spots. Guttate psoriasis often renders these numerous round spots in large areas of the body, such as the trunk, limbs, and scalp. Flexural psoriasis (inverse psoriasis), on the other hand, appears as smooth inflamed patches of skin. Flexural psoriasis occurs in skin folds such as areas around the genitals, the armpits, the overweight stomach, and the breasts. Pustular psoriasis appears as raised bumps and is commonly found locally in the hands and feet, but it can extend to other parts of the body. Erythrodermic psoriasis usually comes with severe itching, swelling, and pain. These radical symptoms may involve the widespread inflammation and exfoliation of the skin. Fingernails and toenails may be affected by nail psoriasis, and often undergo a variety of changes in the appearance. Small indentations in the nails (e.g., pitting), lifting up of the nails, discoloration, thickening, and crumbling of nails may appear due to nail psoriasis. Certain embodiments disclosed herein may be used to treat any type or combination of types of psoriasis, some of which are described above. In certain embodiments, the methods described herein may be used to treat one specific type of psoriasis. In certain alternative embodiments, the methods described herein may be used to treat two or more types of psoriasis.

The severity of psoriasis can be classified or “scored” in a variety of ways. This disease varies from causing relatively minor plaques in a localized area of the body to a generalized psoriasis covering a substantially large area of the body. In a classification method that is based on the surface area of tissue affected, psoriasis can be graded as mild (e.g., affecting less than about 3% of the total area of the body surface (BSA)), moderate (e.g., affecting about 3% to about 10% BSA), or severe (e.g., affecting more than about 10% BSA). By way of comparison, the palm of a person's hand is about 1% BSA. Other scales may also be employed for measuring the severity of psoriasis. For example, in addition to the size of affected or influenced BSA, factors such as the condition duration, the frequency of disease recurrence, disease activity (e.g., degree of plaque redness, thickness, and scaling), response to previous therapies, and the impact of the disease on the person may also be considered to determine the severity of the disease. A subject having less than 3% BSA affected by the condition may be considered to have moderate or severe psoriasis if the affected area is accompanied by radical symptoms such as swelling or pain. A subject having a psoriasis condition that is resistant or recalcitrant to one or more known treatments may also be considered to have severe psoriasis regardless of the size of influenced area. Therefore, psoriasis may be characterized as severe if at least one of the following is observed: the area of influenced tissue is greater than about 10% BSA; the condition (e.g., accompanied by pain and/or swelling) persists for a month or more; the disease activity is substantially active; and the disease is resistant to one or more of known treatments. Psoriasis may also be considered severe if the diseased area comprises between about 10% and about 20% BSA of the subject, between about 20% and about 30% BSA, or greater than about 30% BSA. Certain embodiments disclosed herein may be used to treat psoriasis of various degrees of severity. Some embodiments may be used to treat mild, moderate, or severe psoriasis. Some embodiments may be used to treat any combination of mild, moderate, and severe psoriasis. Some embodiments may be used to treat mild to moderate psoriasis and/or moderate to severe psoriasis.

Severity of psoriasis may be determined according to standard clinical definitions. For example, the Psoriasis Area and Severity Index (PASI) assesses psoriasis disease intensity based on the quantitative assessment of three typical signs of psoriatic lesions: erythema, infiltration, and desquamation, combined with the skin surface area involvement in the four main body areas: head, trunk, upper extremities, and lower extremities. Since its development in 1978, PASI has been used throughout the world by clinical investigators. PASI scores range from 0 (no disease) to 72 (maximum disease), in which higher scores indicate greater disease severity. Improvements in psoriasis are indicated as “PASI 50” (a 50% improvement in PASI from baseline), “PASI 75” (a 75% improvement in PASI from baseline), “PASI 90” (a 90% improvement in PASI from baseline), “PASI 95” (a 95% improvement in PASI from baseline), and “PASI 100” (a 100% improvement in PASI from baseline).

The Physicians Global Assessment (PGA) also assesses psoriasis activity and clinical response to treatment. PGA is a six-point score that summarizes the overall quality (erythema, scaling, and thickness) and extent of plaques relative to the baseline assessment. A patient's response is rated as worse (negative clearance (disease became worse)), poor (0-24% clearance), fair (25-49% clearance), good (50-74% clearance), excellent (75-99% clearance), or cleared (100% clearance).

Other measures of improvement in the disease state of a psoriasis patient may include clinical responses such as the Dermatology Life Quality Index (DLQI), the Short Form 36 Health Survey (SF-36), and the European Quality of Life-5 Dimensions (EQ-5D).

### SUMMARY

The use of three external therapeutic options can treat generalized psoriasis with systemic safety and better efficacy than any systemic or biologic agent: (1) excimer laser phototherapy, for example using XTRAC Velocity®, available from PhotoMedex of Montgomeryville, Pa., and (2) clobetasol spray, for example Clobex®, available from Galderma, S.A. of Cham, Switzerland, and, optionally, (3) calcitriol ointment, for example Vectical®, available from Galderma Pharma, S.A. of Grey, Switzerland. Together, combinations of these treatments may achieve a significant breakthrough result: a PASI 75 response after only four weeks of therapy. Indeed, combinations of these treatments may achieve a PASI 95 to PASI 100 response after only four to six weeks of therapy.

In certain embodiments, a method of treating a skin condition (e.g., psoriasis) comprises: during a first period (e.g., 4 weeks or 1 month), receiving clobetasol propionate spray about twice per day and receiving UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) about twice per week; during a second period (e.g., 4 weeks or 1 month) after the first period, stopping receiving the clobetasol propionate spray and receiving calcitriol ointment about twice per day; during the second period, receiving UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., adjusting (e.g., increasing (e.g., after observing mild erythemal response), decreasing (e.g., after observing plaque thinning, after observing blistering)) phototherapy dosage) about twice per week for at least a portion of the second period; and during a third period (e.g., 4 weeks or 1 month) after the second period, receiving clobetasol propionate spray about twice per day and receiving calcitriol ointment about twice per day.

In certain embodiments, a method of treating a skin condition (e.g., psoriasis) comprises: administering UV phototherapy to a first portion of the skin condition about twice per week during a first period (e.g., 4 weeks or 1 month) in which clobetasol propionate spray is applied to the first portion of the skin condition about twice per day; administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) to the first portion of the skin condition about twice per week during a second period (e.g., 4 weeks or 1 month) in which calcitriol ointment is applied to the first portion of the skin condition about twice per day; administering UV phototherapy to a second portion of the skin condition about twice per week during a third period (e.g., 4 weeks or 1 month) in which clobetasol propionate spray is applied to the second portion of the skin condition about twice per day; and administering UV phototherapy to the second portion of the skin condition about twice per week during a fourth period (e.g., 4 weeks or 1 month) in which calcitriol ointment is applied to the second portion of the skin condition about twice per day. In some embodiments, the method further comprises, during a fifth period (e.g., 4 weeks or 1 month) after the fourth period, receiving clobetasol propionate spray about twice per day and receiving calcitriol ointment about twice per day.

In certain embodiments, a method of treating a skin condition (e.g., psoriasis) comprises: during a first period (e.g., 4 weeks or 1 month), receiving a first non-systemic treatment (e.g., a topical corticosteroid (e.g., clobetasol propionate (e.g., in the form of a spray))) and receiving a second non-systemic treatment (e.g., UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., adjusting (e.g., increasing (e.g., after observing mild erythemal response), decreasing (e.g., after observing plaque thinning, after observing blistering)) phototherapy dosage)); during a second period (e.g., 4 weeks or 1 month) after the first period, receiving a third non-systemic treatment (e.g., a topical Vitamin D analogue (e.g., calcitriol (e.g., in the form of an ointment))); during the second period, receiving the second non-systemic treatment for at least a portion of the second period; and during a third period (e.g., 4 weeks or 1 month) after the second period, receiving the first non-systemic treatment and receiving the third non-systemic treatment. In some embodiments, the first, second, and third non-systemic treatments each have a different mechanism of cellular action.

In certain embodiments, a method of treating a skin condition (e.g., psoriasis) comprises: administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., adjusting (e.g., increasing (e.g., after observing mild erythemal response), decreasing (e.g., after observing plaque thinning, after observing blistering)) phototherapy dosage) about twice per week during a first period (e.g., 4 weeks or 1 month) in which clobetasol propionate spray is applied about twice per day; and administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., adjusting (e.g., increasing (e.g., after observing mild erythemal response), decreasing (e.g., after observing plaque thinning, after observing blistering)) phototherapy dosage) about twice per week during a second period (e.g., 4 weeks or 1 month) in which calcitriol ointment is applied about twice per day.

In certain embodiments, a method of treating a skin condition (e.g., psoriasis) comprises: administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) to a first portion of the skin condition (e.g., about 20% BSA) about twice per week during a first period (e.g., 4 weeks or 1 month) in which clobetasol propionate spray is applied to the first portion of the skin condition about twice per day; administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., adjusting (e.g., increasing (e.g., after observing mild erythemal response), decreasing (e.g., after observing plaque thinning, after observing blistering)) phototherapy dosage) to the first portion of the skin condition about twice per week during a second period (e.g., 4 weeks or 1 month) in which calcitriol ointment is applied to the first portion of the skin condition about twice per day; administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) to a second portion of the skin condition (e.g., comprising BSA affected by the skin condition minus the first portion) about twice per week during a third period (e.g., 4 weeks or 1 month) in which clobetasol propionate spray is applied to the second portion of the skin condition about twice per day; and administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., adjusting (e.g., increasing (e.g., after observing mild erythemal response), decreasing (e.g., after observing plaque thinning, after observing blistering)) phototherapy dosage) to the second portion of the skin condition about twice per week during a fourth period (e.g., 4 weeks or 1 month) in which calcitriol ointment is applied to the second portion of the skin condition about twice per day.

In certain embodiments, a method of treating a skin condition (e.g., psoriasis) comprises: administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) during a first period (e.g., 4 weeks or 1 month) in which a topical corticosteroid (e.g., clobetasol propionate (e.g., in the form of a spray)) is received; and administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., adjusting (e.g., increasing (e.g., after observing mild erythemal response), decreasing (e.g., after observing plaque thinning, after observing blistering)) phototherapy dosage) during a second period (e.g., 4 weeks or 1 month) in which a topical Vitamin D analogue (e.g., calcitriol (e.g., in the form of an ointment)) is received.

In certain embodiments, a method of treating a skin condition (e.g., psoriasis) comprises: administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) to a first portion of the skin condition (e.g., about 20% BSA) during a first period (e.g., 4 weeks or 1 month) in which a topical corticosteroid (e.g., clobetasol propionate (e.g., in the form of a spray)) is applied to the first portion of the skin condition; administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., adjusting (e.g., increasing (e.g., after observing mild erythemal response), decreasing (e.g., after observing plaque thinning, after observing blistering)) phototherapy dosage) to the first portion of the skin condition during a second period (e.g., 4 weeks or 1 month) in which a topical Vitamin D analogue (e.g., calcitriol (e.g., in the form of an ointment)) is applied to the first portion of the skin condition; administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) to a second portion of the skin condition (e.g., comprising BSA affected by the skin condition minus the first portion) during a third period (e.g., 4 weeks or 1 month) in which a topical corticosteroid (e.g., clobetasol propionate (e.g., in the form of a spray)) is applied to the second portion of the skin condition; and administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., adjusting (e.g., increasing (e.g., after observing mild erythemal response), decreasing (e.g., after observing plaque thinning, after observing blistering)) phototherapy dosage) to the second portion of the skin condition during a fourth period (e.g., 4 weeks or 1 month) in which a topical Vitamin D analogue (e.g., calcitriol (e.g., in the form of an ointment)) is applied to the second portion of the skin condition.

In certain embodiments, a method of treating a skin condition (e.g., psoriasis) comprises administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) about thrice per week during a period (e.g., 4 weeks or 1 month) in which clobetasol propionate spray is applied about twice per day.

In certain embodiments, a method of treating a skin condition (e.g., psoriasis) comprises administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) after (e.g., within one day, within one hour) a topical corticosteroid (e.g., clobetasol propionate (e.g., in the form of a spray)) has been applied.

In certain embodiments, a method of treating a skin condition (e.g., psoriasis) comprises administering UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency) at least partially determined based on induration and Fitzpatrick skin phenotype) during a period (e.g., 4 weeks or 1 month) in which a topical corticosteroid (e.g., clobetasol propionate (e.g., in the form of a spray)) is received, wherein after the period, response of the psoriasis is better than response to UV phototherapy alone or clobetasol propionate alone.

In certain embodiments, a method of treating a skin condition (e.g., psoriasis) comprises using a combination of at least two non-systemic treatments (e.g., a topical corticosteroid (e.g., clobetasol propionate (e.g., in the form of a spray)), UV phototherapy (e.g., by directing light propagating from a laser such as an excimer laser, a lamp, an IPL device, or a LED device) (e.g., at a wavelength centered at about 308 nm) (e.g., in an exposure area between about 0.1 cm² and about 10 cm²) (e.g., using an initial dosage between about 300 mJ/cm² and about 900 mJ/cm²) (e.g., adjusting (e.g., increasing (e.g., after observing mild erythemal response), decreasing (e.g., after observing plaque thinning, after observing blistering)) phototherapy dosage after initial dosage) (e.g., using parameters (e.g., dosage, frequency), a Vitamin D analogue (e.g., calcitriol (e.g., in the form on an ointment))), wherein, after a treatment duration of about 12 weeks or less (e.g., about 6 weeks or less), the skin condition has improved by at least about 75% (e.g., at least about 95%).

In certain embodiments, a method comprises instructing any of the methods described herein.

In certain embodiments, a method comprises marketing any of the methods described herein.

For purposes of summarizing the invention and the advantages achieved over the prior art, certain objects and advantages of the invention are described herein. Of course, it is to be understood that not necessarily all such objects or advantages need to be achieved in accordance with any particular embodiment. Thus, for example, those skilled in the art will recognize that the invention may be embodied or carried out in a manner that achieves or optimizes one advantage or group of advantages as taught or suggested herein without necessarily achieving other objects or advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### DETAILED DESCRIPTION

Although certain embodiments and examples are described below, those of skill in the art will appreciate that the invention extends beyond the specifically disclosed embodiments and/or uses and obvious modifications and equivalents thereof. Thus, it is intended that the scope of the invention herein disclosed should not be limited by any particular embodiments described below.

Psoriasis is a chronic condition affecting various tissues including the skin and joints. It occurs, for example, on the skin of the scalp, face, elbows, knees, back, abdomen, knee, hand, foot, and the nails. Psoriasis may also occur on joints of knees, spine, and sacroiliac to cause psoriatic arthritis. The present disclosure is generally related to a treatment of any tissue that has psoriasis symptoms. Although generally described herein as treating the area of diseased tissue, the normal tissue, for example proximate to the diseased tissue (e.g., paralesional tissue), can also be treated. In some embodiments in which the disease is widespread, the treatment may be extended to substantially the entire surface area of the body.

Psoriasis may result from a disorder in the immune system. Research indicates that psoriasis is a T-cell mediated disease and that the skin hyperplasia of the disease is a result of abnormal immune system activation. Injectable biologics are a popular method of treating severe psoriasis. Examples of injectable biologics include: alefacept (e.g., Amevive®, available from Biogen, Inc. of Cambridge, Mass.); etanercept (e.g., Enbrel®, available from Immunex Corporation of Seattle, Wash.); adalimumab (e.g., Humira®, available from Abbott Laboratories of Abbott Park, Ill.); infliximab (e.g., Remicade®, available from Centocor, Inc. of Malvern, Pa.); and ustekinumab (e.g., Stelara®, available from Johnson & Johnson of New Brunswick, N.J.). These biologic medications, while effective in many patients, uniformly work by inducing systemic immunosuppression with increased risk of malignancies, infections including tuberculosis and histoplasmosis, congestive heart failure, lupus-like syndrome, demyelinating diseases, etc. In addition to side effects from systemic immunosuppression, systemic agents can have major organ toxicity as a potential side effect including bone marrow suppression, liver toxicity, kidney toxicity, hypertension, teratogenicity, etc. Moreover, these biologics can have severe side effects such as nausea, fatigue, difficulty sleeping, vomiting, headaches, easy bruising and bleeding, fever, diarrhea, and chills.

Discontinuing biologic treatment or even reducing the doses of the medication causes the psoriasis to revert to its pre-treatment state. Thus, once a patient begins to receive biologics, administration continues for the rest of the patient's life. In some cases, the quantity and/or severity of the psoriasis lesions may become even more severe upon discontinuance or reduction of biologic treatment, which is called a psoriasis “rebound.” Accordingly, once biologic treatment starts, it is never discontinued. Unfortunately, the side-effects of biologic treatment become more severe or likely with prolonged use. In addition, biologics can be very expensive, for example reported to be about $20,000-$70,000 per year. Moreover, since many of the biologic agents are new, there may be some yet unknown serious side effects such as seen with the fatal cases of progressive multifocal leukoencephalopathy (PML) with efalizumab (Raptiva®, formerly available from Genentech, Inc. of South San Francisco, Calif.), which led this biologic to be withdrawn from worldwide use for psoriasis.

The present disclosure generally relates to methods of treating a subject having psoriasis. However, the methods described herein may equally apply to other diseases (e.g., atopic dermatitis, dyshidrosis, eczema, lichen planus, and vitiligo) that are treated on a long-term basis now or in the future and/or to non-human subjects. In some embodiments, psoriasis is treated with a protocol combining a plurality of external, non-biologic, and/or non-systemic therapies during different periods. Such methods may comprise, in series, (1) receiving a first topical and phototherapy, then discontinuing receiving the first topical after a set duration, (2) receiving a second topical and phototherapy, discontinuing receiving the phototherapy after a set duration, and (3) continuing receiving the second topical and again receiving the first topical. Phototherapy may also be used after the set duration for phototherapy, for example to achieve a certain PASI result.

In some embodiments, the protocols described herein may be substantially directed, instructed, prescribed, or marketed by a physician or doctor (e.g., dermatologist) or skin treatment specialist. For example, a doctor may prescribe the topicals for certain durations and/or prescribe phototherapy. In some embodiments, the doctor may administer the phototherapy. In certain embodiments, the doctor may direct or instruct the patient to stop with a drug and/or phototherapy. In some embodiments, the protocols described herein may be substantially directed, instructed, prescribed, or marketed by a topical provider (e.g., drug company). For example, a topical provider may instruct that the topicals should be used for limited durations and/or under certain conditions (e.g., in conjunction with other topicals and/or phototherapy, as part of a treatment protocol, etc.). In some embodiments, the protocols described herein may be substantially directed, instructed, prescribed, or marketed by a phototherapy device provider (e.g., laser company, lamp company, etc.). For example, a phototherapy device provider may instruct that phototherapy should be used for limited durations and/or under certain conditions (e.g., in conjunction with topicals, as part of a treatment protocol, to treat certain BSA or psoriasis severity, etc.). In some embodiments, the protocols described herein may be substantially directed, instructed, prescribed, or marketed by an insurance company. For example, an insurance company may state that it will only reimburse the care provider for topicals and/or phototherapy under certain conditions (e.g., if used as part of a protocol, if some treatments are used in combination with other treatments, if the laser meets certain standards (e.g., ISO certified, UL approved, certified by annual audit, providing failure warnings for expected failure and/or improper dosage delivery, adequate power to timely treat generalized psoriasis, meets performance standards such as a repetition rate of about 400 Hz and/or a maximum fluence per pulse of about 5-8 mJ/cm², etc.), and/or other reasons to safeguard against reimbursement for ineffective treatment). Marketing a protocol may also include touting the effectiveness of a protocol (e.g., in comparison to other protocols) in a way that may entice or induce the actors described herein or others to direct, instruct, or prescribe the protocol. In certain embodiments, the actors described above may direct, instruct, prescribe, or market these protocols to over about 10 patients, over about 100 patients, over about 1,000 patients, over about 10,000 patients, or more, or quantities of patients therebetween.

### Targeted UV Phototherapy

Directed or targeted ultraviolet (UV) phototherapy (e.g., fiberoptically-directed monochromatic UVB light from an excimer laser) is able to target only psoriatic plaques, allowing much more aggressive phototherapy as compared to traditional full-body UVB and PUVA, which exposes non-involved skin as well as psoriatic skin to UV light. With aggressive phototherapy, psoriasis can improve significantly or clear in only approximately ten sessions instead of the thirty to forty sessions needed with regular full-body phototherapy to achieve clearance of the skin. This dramatic difference is attributed to the fact that psoriatic lesions are able to withstand a much higher dose of light than non-involved skin. Targeted phototherapy dosing may be determined by the maximum tolerance of psoriatic skin, whereas traditional full body UV therapy dosing is determined by the MED (minimal erythema dose) of non-involved skin. MED is the traditional limit on how aggressively full-body phototherapy can be conducted since UVB doses beyond the MED will, by definition, blister the patient. Delivery of higher doses than MED are made possible with targeted UV application and results in faster clinical response and much greater clinical efficacy. Supraerythemogenic phototherapy strategy thereby results in faster clinical response and much greater clinical efficacy such that a fewer number of sessions needed for clearance. The supraerythemogenic phototherapy strategy involves delivering UVB at a dose much greater than the MED. Targeted phototherapy has much more efficacy than full-body phototherapy and the onset of action is much faster because the physician can far exceed the patient's MED in aggressiveness of dosimetry. In addition, targeted phototherapy generally results in no photo-damage to non-involved skin, given its targeted application.

Targeted phototherapy has sometimes been viewed as limited to treating mild or moderate psoriasis due to its generally slow nature (e.g., illuminating a spot with a plurality of light pulses until the dosage is achieved, moving the handpiece to a new spot, and repeating). The XTRAC Velocity, available from PhotoMedex of Montgomeryville, Pa. and Carlsbad, Calif., is the latest version of an excimer laser that may be used for targeted phototherapy. The XTRAC Velocity is 300-400% more powerful than its predecessor, the XTRAC® Ultra, also available from PhotoMedex of Montgomeryville, Pa. and Carlsbad, Calif. This increased power makes treatment of generalized, moderate to severe psoriasis not only feasible but attractive because the treatment duration at one spot is reduced (e.g., due to higher pulse frequency, higher energy per pulse, and/or higher average power). The duration of each targeted phototherapy treatment session is decreased to one-third compared to the XTRAC Ultra, which was previously the most powerful phototherapy excimer laser. With XTRAC Velocity, it is expected that generalized psoriasis patients with 10-20% total body surface involvement can be treated in just 10-15 minutes and that great improvement can be achieved after approximately just ten laser treatment sessions.

In addition to the power and treatment time advantages discussed above, XTRAC Velocity also has other advantages over other lasers. Third party annual audits and ISO certification confirm the quality of the XTRAC Velocity. For example, XTRAC Velocity is able to accurately and consistently deliver the set parameters (e.g., within 5-10%), which generally leads to a better clinical outcome. XTRAC Velocity also provides the operator with a warning prior to expected failure as well as warning of failure to have delivered an improper dosage. Safety has been certified by Underwriters Laboratories, Inc. (UL®). Thus, physicians can be confident that the dosages being administered match the treatment being performed and that the laser is safe. Other lasers on the market may over or under dose, have poor reimbursement as a result of shoddy, ineffective treatments, cause physicians and/or patients to lose confidence in phototherapy, lead to accusations of physician malpractice and false billing, etc.

Phototherapy may improve the disease condition in different ways than the biologics described above. For example, certain biologics may suppress the immune system, in particular the activity of T-cells. Certain UV phototherapy may also be able to modify the immune system by regulating T-cell activity. Some phototherapy can further deplete T-cells in psoriatic lesions. Phototherapy has also been shown to decrease Langerhans cells (LC) number, morphology, and function, and this exposure of LC results in suppression of immune response. Such removal of abnormal T-cells may account for, at least in some degree, the generally longer remission period of phototherapy compared to biologics.

While phototherapy has been considered an effective treatment for psoriasis and other skin conditions, some unwanted side effects such as skin cancer and erythema (sunburn) may occur due to, for example, exposure to the intensity of the light. UV phototherapy in some embodiments utilizes light in the UVB band, which extends in wavelength between about 280 nanometers and about 320 nanometers. Psoriasis-afflicted tissue can be effectively rehabilitated with light having wavelengths between about 300 nanometers and about 310 nanometers. Light having a wavelength spectrum between about 295 nanometers and about 325 nanometers can be effective in healing the tissue as well, although certain wavelengths within that range are more effective than other wavelengths within that range. Therefore, to treat the diseased skin and joints of a subject under conditions that can maximize a likelihood of healing of diseased tissue yet that can reduce or minimize risk of erythema and DNA damage (e.g., cancer), a physician may opt for light ranging, for example, between about 295 nanometers and about 320 nanometers, more specifically, between about 300 nanometers and about 310 nanometers, and even more specifically light centered around about 308 nanometers may be preferred.

For effective treatment of skin disorders like psoriasis, the recommended fluence of light having wavelengths distributed between about 300 and about 310 nanometers has been determined to range between about 10 mJ/cm² and about 10 J/cm². More specifically, the fluences preferably range between about 100 mJ/cm² and about 8 J/cm², and between about 300 mJ/cm² and about 3 J/cm². Other dosages are also possible.

In certain embodiments, phototherapy comprises an average power of between about 0.3 watts and about 0.5 watts, between about 2 watts and about 3 watts, between about 2.5 watts and about 4.5 watts, or between about 4.8 watts and about 7.2 watts. Other average powers are also possible. For example, future lasers may be able to develop an average power of 10 watts or even more. In certain embodiments, phototherapy comprises energy between about 8 mJ/pulse and about 15 mJ/pulse or between about 12 mJ/pulse and about 18 mJ/pulse. Other pulse energies are also possible based on the power of the laser and the exposure area.

In certain embodiments, phototherapy comprises a repetition rate of between about 50 pulses/second (Hz) and about 100 Hz (e.g., about 92 Hz), between about 100 Hz and about 500 Hz. More preferably, the repetition rate is between about 125 Hz and about 175 Hz (e.g., about 154 Hz), between about 150 Hz and about 300 Hz (e.g., about 200 Hz, about 225 Hz, about 250 Hz), or between about 350 Hz and about 450 Hz (e.g., about 400 Hz). Other repetition rates are also possible, and higher repetition rates may reduce the treatment duration, for example because the phototherapy device may able to deliver the treatment dosage in a shorter period of time depending on the power per pulse.

In certain embodiments, phototherapy comprises an exposure area (e.g., the area subtended by a single pulse or series of stationary pulses) between about 0.1 cm² and about 10 cm². Some lasers are able to achieve different exposure areas to target different lesions, for example by use of a handpiece attachment or the like. Other exposure areas are also possible. In some embodiments, the exposure area may be related to BSA or lesion size.

In certain embodiments, phototherapy comprises a dosage of about 600 mJ/cm². In certain such embodiments, phototherapy comprises treating an area of about 6,000 cm² or about 30% BSA in less than about 10 minutes (e.g., about 8.1 minutes), treating an area of about 5,000 cm² or about 25% BSA in less than about 7 minutes (e.g., about 6.8 minutes), treating an area of about 4,000 cm² or about 20% BSA in less than about 6 minutes (e.g., about 5.4 minutes), treating an area of about 2,000 cm² or about 10% BSA in less than about 3 minutes (e.g., about 2.7 minutes), treating an area of about 1,000 cm² or about 5% BSA in less than about 2 minutes (e.g., about 1.4 minutes), or treating an area of equal to or less than about 600 cm² or about 3% BSA in less than about 1 minute. Other treatment durations are also possible. For example, the duration for higher dosages may be longer, and the duration for lower dosages may be shorter. It will be appreciated that the values and ranges of certain variables may change based on the values or ranges of other variables, as well as patient condition, etc. Times reduced to less than about 10 to 15 minutes may advantageously help with feasibility of treatment, patient compliance, and/or patient throughput (e.g., the physician may treat more patients due to the reduced time to treat each patient).

Fitzpatrick classifications, for example, can score the individual into six varieties (I to VI) of skin types, ranging from extremely pale-skinned people who are highly susceptible to burns, to extremely dark-skinned people who may suffer serious discoloration from phototherapy. Those with a higher level of skin type such as V-VI are usually prone to an overactive production of melanin following laser skin or hair treatments. This can lead to permanent discoloration or scarring of the skin, and therefore, people with V-VI type skin may undertake any phototherapy with extreme caution. On the other end of the scale, people with skin type I-II are usually pale and likely to experience severe sun damage from exposure to UV light. These skin types are believed to be highly susceptible to skin cancer, and patients are advised to take extreme care to use sunscreen and protect themselves from harmful UV rays. A small percentage of people with skin type experience little to no visible damage from UV light, so skin type alone is not necessarily a sufficient dosage indicator. In some embodiments, dosage of targeted phototherapy may be determined based on a combination of induration (plaque thickness) and Fitzpatrick skin phenotype.

Induration may be measured and/or observed and assigned a score of 1 (mild), 2 (moderate), or 3 (severe). Other induration scoring methods are also possible.

In some embodiments, a physician chooses an initial phototherapy dosage between about 300 mJ/cm² and about 900 mJ/cm² based on induration score and Fitzpatrick skin phenotype and pigmentation of the patient's skin. In contrast to MED, in which the dosage is based on non-affected skin as described below, the dosage is based on the lesion itself. For example, if a patient with type skin has thick plaques, the initial dosage would be higher than a patient with type skin and thin plaques. For another example, if a patient with III-IV type skin has thick plaques, the initial dosage would be higher than a patient with III type skin and thick plaques. An example table of initial dose is provided below, although physicians may adjust the initial dose based on experience, other patient factors, etc.

| Induration Score | Fitzpatrick I-III | Fitzpatrick IV-VI |
|---|---|---|
| 1 | 300 mJ/cm² | 400 mJ/cm² |
| 2 | 500 mJ/cm² | 600 mJ/cm² |
| 3 | 700 mJ/cm² | 900 mJ/cm² |

In some embodiments, phototherapy doses as high as a subject can tolerate are preferred. Dosage may be adjusted based on response to the initial treatment. For example, if the treated area manifests only a mild erythemal response (e.g., light pink), dosage may be increased by about 10% to about 20% or higher (e.g., about 25%) in order to attempt to induce a more potent therapeutic dose in the next treatment. For another example, if the treated area is red, dosage may be increased by about 5% to about 10% to offset effects of patient tolerance build-up (e.g., due to tanning) or to move to a larger, but still tolerable, dose in the next treatment. For another example, if the treated area is blistered, dosage may be decreased by about 10% to about 20% or lower (e.g., about 25%) in order to reduce inflammation in the next treatment. As the psoriasis is treated, the plaque will become thinner (e.g., after about three or four phototherapy treatments), and phototherapy dosage may be reduced accordingly. An example table of dose adjustment is provided below, although physicians may adjust the dosage based on experience, other patient factors, etc.

| Observation | Visible Effect | Dosage Adjustment |
|---|---|---|
| No Effect | No erythema at 12-24 hours | Increase 25% |
| | and no plaque improvement | |
| Minimal Effect | Slight erythema at 12-24 | Increase 15% |
| | hours but no significant | |
| | plaque improvement | |
| Good Effect | Mild to moderate erythema | No Change |
| | response at 12-24 hours | |
| Considerable | Significant improvement | No Change or |
| Improvement | with plaque thinning, reduced | Decrease 15% |
| | scaliness, or pigmentation | |
| Moderate/Severe | Blistering | Reduce 25% |
| Erythema | | |

A minimum (or minimal) erythema dose (MED) corresponds to the minimal dosage at which a noticeable change in the normal or healthy skin color occurs with distinct edges. The amount of energy necessary to induce redness varies from subject to subject and depends on many factors including race, age, and skin color. Consequently, in treating a particular subject, a level of localized exposure equivalent to 1 MED can be determined for the subject. This level of exposure may be characterized by fluence or the amount of energy delivered to a defined area in, e.g., milliJoules per square centimeter (mJ/cm²) or Joules per square centimeter (J/cm²). Diseased tissue is thereby exposed to doses at least as high as that dose which creates a change in color bounded by distinct edges on healthy skin. Recent studies have shown that MED, which is measured on healthy tissue, may not be a good indicator of an appropriate dosage on psoriatic tissue. However, the MED values described herein may be proxies for the dosage (e.g., in mJ/cm²) represented by that MED. Exposure of 1 MED or higher, e.g., doses of about 2 MED to about 8 MED (e.g., about 5 MED), about 2 MED to about 6 MED (e.g., about 4 MED), about 2 MED to about 4 MED (e.g., about 3 MED), about 4 MED to about 8 MED (e.g., about 6 MED), or about 4 MED to about 6 MED (e.g., about 5 MED) may be effective in remedying the diseased condition. Other MEDs are also possible. Dosages higher than 1 MED may be called “supraerythemogenic” phototherapy. The MED dosages described herein may be based on different dosages used throughout a protocol, for example after increases upon tolerance build-up and/or decreases upon plaque thinning.

In a study of nine patients that were retrospectively deemed appropriate candidates for phototherapy and having generalized psoriasis (10-30% BSA) who received supraerythemogenic excimer laser phototherapy using the XTRAC Ultra twice weekly for twelve weeks, seven patients (78%) achieved a PASI 75 response within the twelve weeks, and four patients (44%) achieved a PASI 90 response within 12 weeks. Additionally, after six months with no additional treatment, 83% remained at PASI 50. In a study of eighty patients with stable localized plaque psoriasis covering less than 10% BSA who received supraerythemogenic excimer laser phototherapy using the XTRAC Ultra twice weekly for five weeks, 72% of the patients achieved at least PASI 75 in an average of 6.2 treatments, 84% of the patients achieved at least PASI 75 in 10 or fewer treatments, 35% of the patients achieved at least PASI 90 in an average of 7.5 treatments, and 50% of the patients achieved at least PASI 90 in 10 or fewer treatments. Two patients even achieved PASI 90 in a single treatment. Accordingly, supraerythemogenic UV laser phototherapy can be an effective treatment for psoriasis.

A minimum (or minimal) blistering dose (MBD) corresponds to the minimal dosage at which a noticeable appearance of blistering occurs. The amount of energy necessary to induce blistering varies from subject to subject and depends on many factors including race, age, and skin color. Consequently, in treating a particular subject, a level of localized exposure equivalent to 1 MBD can be determined for the subject. This level of exposure may be characterized by fluence or the amount of energy delivered to a defined area in, e.g., mJ/cm² or J/cm². Diseased tissue is thereby exposed to doses at least as high as that dose that creates blistering on psoriatic skin the next day. For some patients, MBD may correspond to about 3 MED to about 4 MED. Exposure of 1 MBD or higher, e.g., doses of about 2 MBD to about 8 MBD (e.g., about 5 MBD), about 2 MBD to about 6 MBD (e.g., about 4 MBD), about 2 MBD to about 4 MBD (e.g., about 3 MBD), about 4 MBD to about 8 MBD (e.g., about 6 MBD), or about 4 MBD to about 6 MBD (e.g., about 5 MBD) may be effective in remedying the diseased condition. Other MBDs are also possible, for example a large fraction of MBD (e.g., 0.75 MBD, 0.9 MBD, etc.). The MBD dosages described herein may be based on different dosages used throughout a protocol, for example after increases upon tolerance build-up and/or decreases upon plaque thinning. In some embodiments, a dosage of 1 MBD or more may risk secondary infections and/or other side effects. MBD may be less variable from patient-to-patient than MED, and thus treatment dosages defined by a MBD level may be standardized for all patients or a subgroup thereof (e.g., grouped by Fitzpatrick skin type and/or induration).

The determination of phototherapy dosage using MED, MBD, and a combination of induration and Fitzpatrick skin phenotype are discussed herein, but this disclosure is not limited thereto. For example, skin pigmentation and the sensitivity of individual subject can be evaluated by other measurements such as slope of the erythemal dose response curve (sED), baseline pigmentation, and tanning response. Sometimes the patient's subjective tolerance will cause the dosage to be lowered.

In some embodiments, exposing the psoriatic area to high doses of light may treat the condition better than lower dosage light. The number of treatments may be lower with high dose treatments than with low dose treatments. In some embodiments, such reduction in the number of treatments may achieve the clinical result and yet decrease the total quantity or cumulative deposition of UVB light to which skin is exposed. The risk of cancer and skin damage depends on the total number of photons or amount of UVB radiation directed on the skin. Thus, raising the dosage in a single treatment, a couple treatments, or a few treatments can reduce the number of treatments and result in a lower overall number of absorbed photons or UVB radiation, thereby reducing the risk of cancer and other skin damage. Fewer treatments can result in a significant reduction in hyperpigmentation that would normally be experienced due to the body's suntanning response after each treatment. Additionally, lower numbers of treatments may also provide a higher degree of compliance of a subject to an otherwise difficult regimen involving a significant number of visits to the physician. Such a simplification in treatment is favorable, as subjects are less willing or able to adhere to a regimen involving multiple treatments per week for months, years, or a lifetime. As described herein, the high dose treatment of UV may be preferred at least in some embodiments, especially when the number of treatments is reduced, while lower dose treatment may be implemented in some other embodiments. For example, a direct correlation has been also observed between occurrence of blisters and particularly successful treatments. For example, levels of exposure to UV radiation as high as 16 to 20 MED cause UV radiation burns that produce blisters on the skin. Sometimes only a single treatment at this exposure level can rehabilitate the diseased tissue. Thus, employing dosages that cause UV radiation burns accompanied by blistering appears to yield successful single treatment phototherapy. While there is no upper limit to the practical phototherapy dosage, the upper limit of the tolerance of the patient to aggressive phototherapy should be considered. There may be a lower limit to the practical phototherapy dosage because the tissue targeted should be stressed to have an autoimmune response. For example, low dosages that are repeated only after cell turnover may have little or no cumulative effect.

Since high doses of ultraviolet light enhance the risk of skin cancer and erythema as well as cause other skin damage generally associated with premature aging, the extent of a subject's epidermis to which light is directed may be limited. Light is preferably not delivered to regions of skin other than affected areas, which, particularly with psoriasis, are more tolerant than healthy tissue to higher doses of light within the preferred wavelength regions. In treating psoriasis, for example, UV light is preferably directed onto the lesional as well as surrounding paralesional tissue, which although appearing normal is diseased tissue. Treatment, however, should be restricted only to these affected areas of skin, and areas uninvolved are preferably not exposed to the ultraviolet light. The entire body of the patient is generally not to be subjected to the ultraviolet light, especially when high doses of UV light are employed.

In certain embodiments, UV light is delivered to each separate affected region of the body, for example by a handpiece in communication with an excimer laser, as described in U.S. Pat. Nos. 7,144,248 and 7,276,059, each herein incorporated by reference in its entirety. In some embodiments, the ultraviolet light is delivered to each separate affected region of the body, for example by a handpiece in communication with one or more UV lamps (e.g., an excimer lamp, an intense pulsed light (IPL) device, a light emitting diode (LED) device). By avoiding treatment of unaffected portions of skin, the dosage can be raised well above conventional dosages as the affected areas will tolerate substantially higher doses without increased risk of side effects.

In some embodiments, the high doses of UV illumination are directed to an area on the skin that is between about 25 cm² and about 6,000 cm², between about 25 cm² and about 5,000 cm², between about 25 cm² and about 4,000 cm², between about 25 cm² and about 3,000 cm², between about 25 cm² and about 2,000 cm², between about 25 cm² and about 1,800 cm², between about 25 cm² and about 1,600 cm², between about 25 cm² and about 1,400 cm², between about 25 cm² and about 1,200 cm², between about 25 cm² and about 1,000 cm², between about 25 cm² and about 800 cm², between about 25 cm² and about 600 cm², between about 25 cm² and about 500 cm², between about 25 cm² and about 400 cm², between about 25 cm² and about 300 cm², between about 25 cm² and about 200 cm², between about 25 cm² and about 100 cm², between about 25 cm² and about 50 cm², between about 1 cm² and about 25 cm², between about 2,000 cm² and about 5,000 cm², between about 3,000 cm² and about 5,000 cm², between about 4,000 cm² and about 5,000 cm², between about 2,000 cm² and about 6,000 cm², between about 3,000 cm² and about 6,000 cm², between about 4,000 cm² and about 6,000 cm², between about 5,000 cm² and about 6,000 cm², or ranges included therein. In some embodiments, the high doses of UV illumination are directed to an area on the skin that is less than about 6,000 cm², less than about 5,000 cm², less than about 4,000 cm², less than about 3,000 cm², less than about 2,000 cm², less than about 1,800 cm², less than about 1,600 cm², less than about 1,400 cm², less than about 1,200 cm², less than about 1,000 cm², less than about 800 cm², less than about 600 cm², less than about 500 cm², less than about 400 cm², less than about 300 cm², less than about 200 cm², less than about 100 cm², less than about 50 cm², less than about 25 cm², or values therebetween.

In some embodiments, the high doses of UV illumination are directed to an area on the skin that is between about 1% BSA and about 30% BSA, between about 1% BSA and about 25% BSA, between about 1% BSA and about 20% BSA, between about 1% BSA and about 15% BSA, between about 1% BSA and about 10% BSA, between about 1% BSA and about 5% BSA, between about 3% BSA and about 30% BSA, between about 3% BSA and about 25% BSA, between about 3% BSA and about 20% BSA, between about 3% BSA and about 15% BSA, between about 3% BSA and about 10% BSA, between about 3% BSA and about 5% BSA, between about 10% BSA and about 30% BSA, between about 10% BSA and about 25% BSA, between about 10% BSA and about 20% BSA, between about 10% BSA and about 15% BSA, or ranges included therein. In some embodiments, the high doses of UV illumination are directed to an area on the skin that is less than about 30% BSA, less than about 25% BSA, less than about 20% BSA, less than about 15% BSA, less than about 10% BSA, less than about 5% BSA, less than about 3% BSA, less than about 1% BSA, or values therebetween. There is no theoretical limit to the BSA level that may be treated with phototherapy, but there may be practical limits such as patient tolerance and time. As lasers become more powerful and faster (e.g., like the XTRAC Velocity), these practical limits may be moot.

The temporal extent over which exposure occurs may also be of interest. Exposure of the affected area to the UV light may result in heating of the tissue. Unless this heat is sufficiently dissipated, thermal blistering may result. In some embodiments, the high dose of energy provided by the UV light is distributed over a certain length of time. This duration of exposure may, for example, range between about 100 milliseconds and about 1,500 milliseconds for radiation delivered at about 308 nanometers. The illumination may be within a short enough time to be practical, yet long enough to reduce, mitigate, or prevent blistering caused by thermal overload. Certain lasers such as the XTRAC Velocity and the XTRAC Ultra may advantageously cause heating of the skin by only about 5° C., and may be considered “cold” lasers not inducing significant heating, reducing inflammation, reducing pain, and/or accelerating healing.

In some embodiments, an excimer laser can be employed to generate short high power pulses of light having a wavelength of about 308 nanometers. These pulses can be high in peak power, e.g., about half a million watts, but short in duration, for example, lasting much less than about 100 nanoseconds (e.g., about 30 nanoseconds). The laser, however, may produce a plurality of such pulses at a repetition rate of about 100, 150, 200, 250, 300, 400, 450, or 500 Hz, and ranges therebetween. Tissue exposed to a plurality of these short pulses will increase in temperature slightly with application of each pulse. The cumulative effect of the plurality of pulses to raise the temperature of the tissue to a certain amount depends in part on the heat capacity of the tissue. The energy from the laser may be spread over a long enough period of time so as to permit sufficient dissipation to avoid excessive build-up of heat from the plurality of short pulses. Thermal damage caused by raising the temperature of the skin above, for example, the blister temperature of 50° C., can thereby be reduced, mitigated, or prevented. The duration of exposure of the affected tissue to the therapeutic doses of UV light, however, depends on the particular dose level. In some embodiments, UV lamps (including UV excimer lamps like the VTRAC® excimer lamp system, available from PhotoMedex of Montgomeryville, Pa.), intense pulsed light (“IPL”) devices, light-emitting diode (“LED”) devices (e.g., available from Photo Therapeutics, Ltd. of Altrincham, United Kingdom and Photo Therapeutics, Inc. of Carlsbad, Calif.), or other phototherapy devices available now or developed in the future can be employed to generate the UV light.

In some embodiments, phototherapy is administered or received without any help from a light-sensitizing agent. In some embodiments, light-sensitizing agents may be used, for example to increase the sensitivity of a cell to UV. In certain such embodiments, one or more light-sensitizing agents may be applied to the subject or received by the subject before or after phototherapy. Examples of light-sensitizing agents include, but are not limited to, coal tar, psoralen, acitretin, and salicylic acid.

Topical Agents such as Corticosteroids

Although phototherapy is beneficial for treating psoriasis, topical drugs have also been used. Examples of topical treatments include dithranol/anthralin, coal tar, corticosteroids (e.g., clobetasol propionate, desoximetasone, fluocinonide), vitamins (e.g., Vitamin D analogues such as calcitriol, calcipotriol, taxarotene, and calcipotriene; retinoids such as retinol, retinal, tretinoin, isotretinoin, alitretinoin, etretinate, acitretin, tazarotene, bexarotene, and adapalene), pimecrolimus (e.g., Elidel®, available from Novartis of Basel, Switzerland), tacrolimus (e.g., Prograf®, available from Astellas Pharma, Inc. of Tokyo, Japan), and argan oil. Anthralin may disadvantageously stain clothes and skin and irritate skin. Coal tar may disadvantageously have an unpleasant odor, cause skin irritation, and may be linked with skin cancer. The long-term safety of some vitamins such as taxarotene and calcipotriene is not well known. Prolonged use of corticosteroids may cause thinning of the skin, striae, masking of local infections, hypopigmentation, and steroidal effects.

In certain protocols described herein, a first topical, external drug, or non-systemic treatment is used in conjunction with UV phototherapy, for example at the inception of the protocol or during the entire protocol. A protocol may comprise administering UV phototherapy during a period in which a first topical is applied or received. In some embodiments, the period comprises about four weeks or one month (e.g., based on the duration that the first topical can be used without causing serious systemic effects). In some embodiments, the period comprises less than about one week, greater than about four weeks, about one week, two weeks, three weeks, four weeks, or ranges therebetween. The first topical is different from the light-sensitizing agents discussed above. In some embodiments, the first topical comprises a corticosteroid. In some embodiments, the corticosteroid comprises clobetasol propionate. Clobetasol propionate is sold worldwide under trade names such as Butavate, Clobesol, Clobex, Cosvate, Dermatovate, Dermovate, Embeline, Movate, Novate, Olux, Temovate, and Tenovate, in forms such as ointment, emollient, foam, cream, lotion, gel, shampoo, and spray. Clobex spray is able to cover large surface areas in a more efficient manner than ointment, emollient, foam, cream, lotion, gel, shampoo, etc. and thus may be more tolerated by patients with severe psoriasis needing to receive the drug about twice daily. Clobex spray comprises the active ingredient 0.05% clobetasol propionate. Inactive ingredients include isopropyl myristate, alcohol, undecylenic acid, and sodium lauryl sulfate. The spray mechanism with which Clobex spray received Food and Drug Administration approval is specially engineered to apply the correct dose. Clobex spray delivers clobetasol propionate in a matrix of alcohol, which rapidly evaporates from the skin and leaves the clobetasol propionate in immediate contact with the skin. Clobex is currently indicated for use on 20% BSA or less; future regulatory approvals may increase the allowed BSA. Other forms of clobetasol propionate (e.g., ointment, emollient, foam, cream, lotion, gel, shampoo, etc.) may also be used, for example when the psoriasis is mild to moderate. Some of these other forms of clobetasol propionate or other first topicals may also be indicated for use on greater than 20% BSA.

Aggressive phototherapy can cause skin inflammation, particularly as phototherapy dosages increase. Certain topicals such as clobetasol propionate spray can treat inflammation, and thus may allow higher dosages of phototherapy to be used. As described above, higher phototherapy doses may advantageously lead to faster treatment and longer remission periods. Thus, the combination of phototherapy and certain topicals such as clobetasol propionate may lead to faster treatment than phototherapy alone.

In addition, certain topicals such as clobetasol propionate can alone treat the psoriasis. In a study of 1,254 subjects treated with clobetasol propionate spray 0.05% as monotherapy twice daily for four weeks, 35.7% of patients achieved clearance (PASI 100) and 37.3% of patients were almost clear using a 6-point target plaque severity scale. Additionally, at week 4, 80% of subjects achieved target plaque severity (TPS) success, p<0.001. This was specifically defined as a score of clear, almost clear using the TPS scale, or an improvement in severity of two grades.

While the precise mechanism of clobetasol propionate is unknown, it is believed to be the induction of phospholipase A₂ inhibitory proteins (lipocortins), which may control the biosynthesis of potent mediators of inflammation such as prostaglandins and leukotrienes by inhibiting the release of common precursor arachidonic acid, which is released from membrane phospholipids by phospholipase A₂. Other topicals such as corticosteroids other than clobetasol propionate that can provide relief of inflammatory and pruritic manifestations of corticosteroid-responsive dermatoses or via other cellular mechanisms may also be used with protocols described herein.

Topical corticosteroids have the potential for adrenal suppression. However, when topical steroids are used for one month or less at a time, which is within FDA guidelines, this transient adrenal suppression that may otherwise result is not a clinical problem. In certain embodiments in which the first topical comprises a corticosteroid such as clobetasol propionate spray, patients are instructed to cease use of the first topical after one month or four weeks or less, thereby providing a steroid-free interval after four weeks. In some embodiments, the first topical is received about twice daily (e.g., substantially twice daily or exactly twice daily). Other first topical application frequencies are also possible. For example, the first topical may be received about once daily, about thrice daily, about one to three times daily, about one to four times daily, about two to four times daily, about every other day, a certain number of times (e.g., 1, 2, 3, 4, 5, 6, 7, 8, 9, 10, 11, 12, 13, 14, 15, 16, 17, 18, 19, 20, 21, and ranges therebetween) over the span of several days (e.g., about every 1 day, about every 2 days, about every 3 days, about every 4 days, about every 5 days, about every 6 days, about every 7 days, and ranges therebetween), a certain number of times (e.g., 1, 2, 3, 4, 5, 6, 7, 8, 9, 10, 11, 12, 13, 14, 15, 16, 17, 18, 19, 20, 21, and ranges therebetween) over the span of a week, a certain number of times (e.g., 7, 14, 21, 28, 35, 42, 49, 56, 63, 70, 77, 84, 91, 98, 105, 112, and ranges therebetween) over the span of several weeks (e.g., about 1 week, about 2 weeks, about 3 weeks, about 4 weeks), a certain number of times (e.g., 7, 14, 21, 28, 35, 42, 49, 56, 63, 70, 77, 84, 91, 98, 105, 112, and ranges therebetween) over the span of a protocol period (e.g., an initial period, an intermediate period, a final period), or a certain number of times (e.g., 28, 56, 84, 112, 140, 168, 196, 224, 252, and ranges therebetween) over the span of a protocol. Because patients may, e.g., accidentally miss one to all applications in a day or multiple days or receive too many applications in a day or multiple days, the values described herein may be the statistical mode or mean over the span of 2 days, several days, a week, several weeks, or a treatment period. Accordingly, the first topical may be received, on average, once daily, thrice daily, one to three times daily, one to four times daily, two to four times daily, every other day, a certain number of times (e.g., 1, 2, 3, 4, 5, 6, 7, 8, 9, 10, 11, 12, 13, 14, 15, 16, 17, 18, 19, 20, 21, and ranges therebetween) over the span of several days (e.g., about every 1 day, about every 2 days, about every 3 days, about every 4 days, about every 5 days, about every 6 days, about every 7 days, and ranges therebetween), a certain number of times (e.g., 1, 2, 3, 4, 5, 6, 7, 8, 9, 10, 11, 12, 13, 14, 15, 16, 17, 18, 19, 20, 21, and ranges therebetween) over the span of a week, a certain number of times (e.g., 7, 14, 21, 28, 35, 42, 49, 56, 63, 70, 77, 84, 91, 98, 105, 112, and ranges therebetween) over the span of several weeks (e.g., about 1 week, about 2 weeks, about 3 weeks, about 4 weeks), a certain number of times (e.g., 7, 14, 21, 28, 35, 42, 49, 56, 63, 70, 77, 84, 91, 98, 105, 112, and ranges therebetween) over the span of a protocol period (e.g., an initial period, an intermediate period, a final period), or a certain number of times (e.g., 28, 56, 84, 112, 140, 168, 196, 224, 252, and ranges therebetween) over the span of a protocol. The dosage of the first topical may be adjusted based on the species, concentration, physical form, etc. of the first topical. The first topical may be a corticosteroid or other drug available now or developed in the future.

In some embodiments, phototherapy combined with the first topical is received about twice weekly (e.g., substantially twice weekly or exactly twice weekly). The dosage and/or frequency of the phototherapy may be adjusted as described herein, based on PASI response, patient tolerance, patient availability, etc. For example, the phototherapy may be administered or received every other day, a certain number of times (e.g., 1, 2, 3, 4, and ranges therebetween) over the span of several days (e.g., about every 1 day, about every 2 days, about every 3 days, about every 4 days, about every 5 days, about every 6 days, about every 7 days, and ranges therebetween), a certain number of times (e.g., 1, 2, 3, 4, and ranges therebetween) over the span of a week, a certain number of times (e.g., 1, 2, 3, 4, 5, 6, 7, 8, 9, 10, 11, 12, 13, 14, 15, 16, 17, 18, 19, 20, 21, 28, 35, 42, 49, 56, 63, 70 and ranges therebetween) over the span of several weeks (e.g., about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 16 weeks, about 20 weeks), a certain number of times (e.g., 1, 2, 3, 4, 5, 6, 7, 8, 9, 10, 11, 12, 13, 14, 15, 16, 17, 18, 19, 20, 21, and ranges therebetween) over the span of a protocol period (e.g., an initial period, an intermediate period, a final period), or a certain number of times (e.g., 1, 2, 3, 4, 5, 6, 7, 8, 9, 10, 11, 12, 13, 14, 15, 16, 17, 18, 19, 20, 21, 28, 35, 42, and ranges therebetween) over the span of a protocol. The protocols herein discuss optional phototherapy administration periods, and it will be appreciated that phototherapy can be administered or received for 1 week, 2 weeks, 3 weeks, 4 weeks, 5 weeks, 6 weeks, 7 weeks, 8 weeks, 9 weeks, 9 weeks, 10 weeks, 11 weeks, 12 weeks, 16 weeks, 20 weeks, and ranges therebetween.

Possible advantages of using phototherapy and clobetasol propionate in combination during a treatment period include, for example: (1) the likely possibility of synergistic efficacy where two very effective, external therapeutic modalities are combined to possibly result in patients (e.g., 100% of patients) achieving PASI 75 by week 4 of therapy (rather than by week 12 of therapy), a level of efficacy that would be unequalled in terms of therapeutic effect; (2) it can be tedious to treat multiple small plaques with targeted UV phototherapy alone, and the use of clobetasol propionate spray can help eliminate such small recalcitrant psoriatic plaques en masse; (3) UV phototherapy can help eliminate stubborn plaques that are still resistant to clobetasol propionate spray used twice daily for one month; and (4) clobetasol propionate has a different mechanism of cellular action than phototherapy and thus targets the disease in a different manner, likely leading to faster clearance times and longer remission periods than clobetasol propionate alone or UV phototherapy alone.

Treatment using a variety of cellular mechanisms may provide synergy with respect to eliminating the effects of the disease, treating the cause of the disease, and producing normal cellular function. An example of two different cellular mechanisms includes: (1) clobetasol propionate may be used to reduce inflammation, to capitalize on the fact that cells with reduced inflammation may be less prone to proliferate; and (2) UV phototherapy may be used to destroy psoriatic lesions and ward off cells displaying psoriatic proliferation.

Clobetasol propionate may be used following phototherapy, for example in order to avoid possible interference of the phototherapy by the clobetasol propionate, its carrier agent, or other inactive ingredients and/or to treat inflammation caused by the phototherapy. For example, in embodiments in which clobetasol propionate is received about twice daily, the patient may be instructed to adjust the application schedule such that clobetasol propionate is not received at least about 12 hours, at least about 15 hours, or at least about 18 hours prior to scheduled phototherapy visits.

Clobetasol propionate may also be used before phototherapy, for example in order to reduce the erythemal response of the treated tissue. Some forms of clobetasol propionate (e.g., Clobex spray) do not significantly absorb UV light (e.g., absorption less than 90%) and do not significantly interfere with UV phototherapy. In some embodiments, the clobetasol propionate may be received less than about one day, less than about two hours, less than about one hour, or less than about one hour before the phototherapy in order to allow the solvent for the clobetasol propionate time to evaporate and/or for the clobetasol propionate time to affect the tissue (e.g., reducing the erythemal response). In some embodiments in which clobetasol propionate is received prior to phototherapy, the dosage of the phototherapy may be adjusted (e.g., by about 10%, by the amount expected to be absorbed by the clobetasol propionate, etc.) to compensate for possible absorption by the clobetasol propionate.

In some embodiments, a systemic biologic (e.g., comprising alefacept, etanercept, adalimumab, infliximab, ustekinumab, and/or efalizumab), rather than a topical corticosteroid such as clobetasol propionate, may be combined with phototherapy. The combination therapy is likely to lead to better PASI response and/or longer remission than a systemic biologic alone or phototherapy alone. The combination with phototherapy may even allow the patient to stop using the systemic biologic under certain circumstances, for example in combination with a topical corticosteroid. Protocols including use of systemic biologics followed by phototherapy may also be possible, for example as described in U.S. Patent Pub. No. 2011/0002918, incorporated herein by reference in its entirety, alone or in combination with a topical corticosteroid, Vitamin D analogue, etc.

### FIRST EXAMPLE PROTOCOL

The first example protocol uses a rapidly effective agent first (e.g., combination clobetasol propionate and UV phototherapy), and then transitions to a safer maintenance agent (e.g., UV phototherapy alone). With this protocol and other protocols described herein in which systemic biologics are not used, the concerns about lymphoma and other internal cancers, tuberculosis and histoplasmosis, congestive heart failure, demyelinating disorders, etc. are eliminated along with traditional systemic risks such as hepatotoxicity, renal toxicity, and bone marrow suppression. Moreover, instead of spending hours of uncompensated staff time trying to obtain authorization for costly biologic agents, clinicians can be well reimbursed for conducting phototherapy procedures.

The first example protocol includes two distinct periods: Period A, four weeks in duration, and Period B, two weeks in duration. This protocol combines Clobex spray with XTRAC Velocity targeted UV phototherapy as the initial treatment of generalized plaque psoriasis, followed by maintenance phototherapy.

| Period | Week | Clobex ® | XTRAC ® Velocity |
|---|---|---|---|
| A | 1 | 2/d | 2/wk |
| | 2 | 2/d | 2/wk |
| | 3 | 2/d | 2/wk |
| | 4 | 2/d | 2/wk |
| B | 5 | | 2/wk |
| | 6 | | 2/wk |

### Period A—Weeks 1-4

| Period | Week | Clobex ® | XTRAC ® Velocity |
|---|---|---|---|
| A | 1 | 2/d | 2/wk |
| | 2 | 2/d | 2/wk |
| | 3 | 2/d | 2/wk |
| | 4 | 2/d | 2/wk |

During Period A (weeks 1 through 4), patients receive Clobex spray about twice daily (e.g., substantially twice daily or exactly twice daily) and patients are administered or receive UV phototherapy treatments about twice weekly (e.g., substantially twice weekly or exactly twice weekly), for example with the XTRAC Velocity. The goal of Period A may be to achieve PASI 75 within four weeks. Other response goals and times are also possible. For example, the goal of Period A may be to achieve PASI 50, PASI 75, PASI 90, PASI 95, or PASI 100, and ranges therebetween, within about 1 week, about 2 weeks, about 4 weeks, and ranges therebetween, and combinations thereof. Administration, application, or receipt of the Clobex spray may be performed by the patient (e.g., at home). For example, receipt of the Clobex spray or other topicals described herein may be as a result of the physician prescribing Clobex spray and the patient applying the Clobex spray. UV phototherapy administration, application, or receipt is currently generally performed at the physician's office since home lasers are generally not available and since patients are generally not trained in laser use.

For the entire 6 weeks of the protocol, laser treatments are preferably at least one day apart (e.g., in some embodiments they do not occur on consecutive days in order to gauge effects of the phototherapy). If the patient misses a laser treatment, the patient can make up that treatment within the week.

In the patient's first visit to the physician, the initial PASI and/or PGA may be calculated. The patient is provided with Clobex spray or a prescription therefor to be used twice daily during Period A. As described herein, a topical corticosteroid other than Clobex may be used, for example comprising clobetasol propionate or another active ingredient, in the form of a spray or other topical such as ointment, emollient, foam, cream, lotion, gel, shampoo, etc. Although the Clobex dosage of the example protocol is twice daily, other dosages may be used depending on the corticosteroid used. For example, a more dilute clobetasol propionate may be used more often or a more concentrated clobetasol propionate may be used less often. Other corticosteroids may have different prescribed treatment dosages.

During the patient's first visit to the physician, the physician may also determine the appropriate laser dosage, for example based on a combination of induration and Fitzpatrick skin phenotype, and administer the initial laser treatment. As described herein, phototherapy other than using the XTRAC Velocity may be used, for example comprising another excimer laser or another UV light source. Although the frequency of the phototherapy is twice weekly, other frequencies may be used depending on, for example, the light source used, the patient's ability to withstand the phototherapy, and/or the patient's response to the phototherapy and/or the Clobex. Additionally during the first visit, the physician may perform a physical exam, ask the patient to fill out a quality of life survey, and take photographs of the patient's front and back.

As the patient already has the Clobex medication, subsequent visits may be limited to XTRAC Velocity treatments and review of progress, side effects, etc. The dosage of each laser treatment may be adjusted as patient tolerability to higher dosages becomes more certain, for example increasing by a small amount each treatment, as otherwise described herein, or based on other criteria. During a week 4 visit, the physician may again ask the patient to fill out a quality of life survey.

### Period B—Weeks 5-6

| Period | Week | Clobex ® | XTRAC ® Velocity |
|---|---|---|---|
| B | 5 | | 2/wk |
| | 6 | | 2/wk |

During Period B (weeks 5 and 6), patients are administered or receive XTRAC Velocity treatments about twice weekly (e.g., substantially twice weekly or exactly twice weekly) for a total of twelve XTRAC Velocity treatments during the protocol. As described herein, the dosages of the phototherapy treatments may be adjusted during each treatment. Patients do not receive Clobex spray during Period B, which is a steroid-free interval of the protocol. The goal of Period B may be to maintain the patient's response using only non-steroid topical options.

Week 5 (2 Visits)

In the patient's first visit to the physician in week 5, the PASI and/or PGA may be calculated. The patient is provided with clear instructions about ceasing use of any Clobex during Period B that may be left over from Period A.

The physician also administers an XTRAC Velocity treatment during the first visit in week 5. As described herein, the dosages of the phototherapy treatments may be adjusted during each treatment. Additionally during the first visit in week 5, the physician may perform a physical exam, confirm adherence to the protocol, ask about side effects, take photographs of the patient's back and front, and reconcile new medications taken by the patient. The patient's second visit in week 5 may be limited to XTRAC Velocity treatment and review of progress, side effects, etc.

Week 6 (2 Visits)

The patient's first visit to the physician in week 6 may be limited to XTRAC Velocity treatment and review of progress, side effects, etc. As described herein, the dosages of the phototherapy treatments may be adjusted during each treatment. In the patient's second visit to the physician in week 6, the PASI and/or PGA may be calculated to determine if the patient will continue XTRAC Velocity treatment past week 6.

In the final or termination visit, the PASI and/or PGA may be calculated and a quality of life survey may be filled out. Additionally during the termination visit, the physician may perform a physical exam, confirm adherence to the protocol, ask about side effects, take photographs of the patient's back and front, reconcile new medications taken by the patient, and complete protocol paperwork.

### Results

While synergistic effect was expected, the clinical results using a combination of UV phototherapy and clobetasol propionate are even better than might have been expected. Patients have achieved PASI 95 to PASI 100 after just four weeks treatment using a combination of phototherapy and clobetasol propionate and the last two weeks of which used phototherapy alone or in combination with a second topical as described herein. The combination of clobetasol propionate and UV phototherapy clearly has a synergistic effect that can achieve better results than clobetasol propionate alone or UV phototherapy alone. By way of comparison, studies of treatment using systemic biologic Stelara in 45 mg doses achieved only 67%-68% of patients achieving PASI 75 after 12 weeks, studies of treatment using systemic biologic Stelara in 90 mg doses achieved only 66%-76% of patients achieving PASI 75 after 12 weeks, and studies of treatment using systemic biologic Enbrel in 50 mg doses achieved only 49%-57% of patients achieving PASI 75 after 12 weeks. Moreover, there is little to no risk of systemic side effects such as lymphoma and other internal cancers, tuberculosis and histoplasmosis, congestive heart failure, demyelinating disorders, etc. are eliminated along with traditional systemic risks such as hepatotoxicity, renal toxicity, and bone marrow suppression. By contrast, treatment with systemic biologics risks all of those side effects and, once taken by a patient, generally cannot be stopped. Increasing the aggressiveness of the phototherapy, for example from about twice weekly to about thrice weekly, may further reduce the treatment duration.

### SECOND EXAMPLE PROTOCOL

The second example protocol uses a rapidly effective agent (e.g., combination clobetasol propionate and UV phototherapy). The second example protocol includes one period: Period A, which is four weeks in duration. This protocol combines Clobex spray with an aggressive schedule of XTRAC Velocity targeted UV phototherapy as the treatment of generalized plaque psoriasis.

| Period | Week | Clobex ® | XTRAC ® Velocity |
|---|---|---|---|
| A | 1 | 2/d | 3/wk |
| | 2 | 2/d | 3/wk |
| | 3 | 2/d | 3/wk |
| | 4 | 2/d | 3/wk |

During Period A (weeks 1 through 4), patients receive Clobex spray about twice daily (e.g., substantially twice daily or exactly twice daily) and patients are administered or receive UV phototherapy treatments about thrice weekly, for example with the XTRAC Velocity. Administration, application, or receipt of the Clobex spray may be performed by the patient (e.g., at home). For example, receipt of the Clobex spray or other topicals described herein may be as a result of the physician prescribing Clobex spray and the patient applying the Clobex spray. UV phototherapy administration, application, or receipt is currently generally performed at the physician's office since home lasers are generally not available and since patients are generally not trained in laser use.

For the entire 4 weeks of the protocol, laser treatments are preferably at least one day apart (e.g., in some embodiments they do not occur on consecutive days in order to gauge effects of the phototherapy). If the patient misses a laser treatment, the patient can make up that treatment within the week. Due to the more aggressive phototherapy schedule of the second example protocol, patients should be highly encouraged to not miss any visits.

In the patient's first visit to the physician, the initial PASI and/or PGA may be calculated. The patient is provided with Clobex spray or a prescription therefor to be used twice daily during Period A. As described herein, a topical corticosteroid other than Clobex may be used, for example comprising clobetasol propionate or another active ingredient, in the form of a spray or other topical such as ointment, emollient, foam, cream, lotion, gel, shampoo, etc. Although the Clobex dosage of the example protocol is twice daily, other dosages may be used depending on the corticosteroid used. For example, a more dilute clobetasol propionate may be used more often or a more concentrated clobetasol propionate may be used less often. Other corticosteroids may have different prescribed treatment dosages.

During the patient's first visit to the physician, the physician may also determine the appropriate laser dosage, for example based on a combination of induration and Fitzpatrick skin phenotype, and administer the initial laser treatment. As described herein, phototherapy other than using the XTRAC Velocity may be used, for example comprising another excimer laser or another UV light source. Although the frequency of the phototherapy is thrice weekly, other frequencies may be used depending on, for example, the light source used, the patient's ability to withstand the phototherapy, and/or the patient's response to the phototherapy and/or the Clobex (e.g., seven times every two weeks). Additionally during the first visit, the physician may perform a physical exam, ask the patient to fill out a quality of life survey, and take photographs of the patient's front and back.

As the patient already has the Clobex medication, subsequent visits may be limited to XTRAC Velocity treatments and review of progress, side effects, etc. The dosage of each laser treatment may be adjusted as patient tolerability to higher dosages becomes more certain, for example increasing by a small amount each treatment, as otherwise described herein, or based on other criteria.

In the final or termination visit, the PASI and/or PGA may be calculated and a quality of life survey may be filled out. Additionally during the termination visit, the physician may perform a physical exam, confirm adherence to the protocol, ask about side effects, take photographs of the patient's back and front, reconcile new medications taken by the patient, and complete protocol paperwork.

Topical Agents such as Vitamin D Analogues

In certain protocols described herein, a second topical may be used in conjunction with phototherapy after the use of the first topical has ceased. The second topical can maintain the effect of the first topical. For example, the effect of clobetasol propionate spray can be maintained with a safe, non-steroidal topical medication, such as calcitriol. Vectical ointment comprises the active ingredient 3 μg/g calcitriol. Inactive ingredients include mineral oil, dl-α-tocopherol, and white petrolatum. This newer Vitamin D analogue can be used up to 200 grams (g) weekly, which can cover much greater BSA when compared to prior Vitamin D analogues such as topical calcipotriol (e.g., Dovonex® and Taclonex®, both available from Leo Pharma of Ballerup, Denmark), which is limited to 100 g weekly, due to the better safety profile of calcitriol. For example, Dovonex may cause hypercalcemia if overused. Although patients are likely to have PASI 75 or better after ceasing using a combination of the first topical and phototherapy, a second topical such as calcitriol can maintain the effect of the first topical. Some calcitriol ointments can be used for patients with generalized plaque psoriasis covering up to 35% BSA. By contrast, the limited dosage of calcipotriol and calcipotriene may limit the treatable BSA.

In certain protocols described herein, calcitriol is used in conjunction with phototherapy after use of clobetasol propionate has ceased. Calcitriol is sold worldwide under trade names such as Calcijex, Rocaltrol, and Vectical, in forms such as ointment, emollient, foam, cream, lotion, gel, and shampoo. Other Vitamin D analogues (e.g., calcipotriol, cholecalciferol, tacalcitol, etc.) are also possible, although dosage should be adjusted appropriately. Vitamin D analogues have a different mechanism of cellular action than phototherapy and corticosteroids, and thus target the disease in yet another different manner. For example, Vitamin D analogues may interact with Vitamin D₃ receptors to induce cells to make proteins that slow the growth rate and/or that blunt the activity of inflamed cells. Calcitriol may also help to heal skin damaged by topical corticosteroids (e.g., clobetasol propionate) and phototherapy, for example improving the integrity of the stratum corneum and improving recovery of the epidermal barrier that can control water loss and prevent infections.

In a comparative study in 250 patients, calcitriol caused significantly less skin irritation as compared to calcipotriol. With the availability of calcitriol, a Vitamin D analogue can be used to treat patients with generalized psoriasis. In some embodiments, the second topical is applied about twice daily (e.g., substantially twice daily or exactly twice daily). Other second topical application frequencies are also possible. For example, the second topical may be applied about once daily, about thrice daily, about one to three times daily, about one to four times daily, about every other day, about two to four times daily, a certain number of times (e.g., 1, 2, 3, 4, 5, 6, 7, 8, 9, 10, 11, 12, 13, 14, 15, 16, 17, 18, 19, 20, 21, and ranges therebetween) over the span of several days (e.g., about every 1 day, about every 2 days, about every 3 days, about every 4 days, about every 5 days, about every 6 days, about every 7 days, and ranges therebetween), a certain number of times (e.g., 1, 2, 3, 4, 5, 6, 7, 8, 9, 10, 11, 12, 13, 14, 15, 16, 17, 18, 19, 20, 21, and ranges therebetween) over the span of a week, a certain number of times (e.g., 7, 14, 21, 28, 35, 42, 49, 56, 63, 70, 77, 84, 91, 98, 105, 112, and ranges therebetween) over the span of several weeks (e.g., about 1 week, about 2 weeks, about 3 weeks, about 4 weeks), a certain number of times (e.g., 7, 14, 21, 28, 35, 42, 49, 56, 63, 70, 77, 84, 91, 98, 105, 112, and ranges therebetween) over the span of a protocol period (e.g., an initial period, an intermediate period, a final period), or a certain number of times (e.g., 28, 56, 84, 112, 140, 168, 196, 224, 252, and ranges therebetween) over the span of a protocol. Because patients may, e.g., accidentally miss one to all applications in a day or multiple days or receive too many applications in a day or multiple days, the values described herein may be the statistical mode or mean over the span of 2 days, several days, a week, several weeks, or a treatment period. Accordingly, the second topical may be applied, on average, once daily, thrice daily, one to three times daily, one to four times daily, every other day, two to four times daily, a certain number of times (e.g., 1, 2, 3, 4, 5, 6, 7, 8, 9, 10, 11, 12, 13, 14, 15, 16, 17, 18, 19, 20, 21, and ranges therebetween) over the span of several days (e.g., about every 1 day, about every 2 days, about every 3 days, about every 4 days, about every 5 days, about every 6 days, about every 7 days, and ranges therebetween), a certain number of times (e.g., 1, 2, 3, 4, 5, 6, 7, 8, 9, 10, 11, 12, 13, 14, 15, 16, 17, 18, 19, 20, 21, and ranges therebetween) over the span of a week, a certain number of times (e.g., 7, 14, 21, 28, 35, 42, 49, 56, 63, 70, 77, 84, 91, 98, 105, 112, and ranges therebetween) over the span of several weeks (e.g., about 1 week, about 2 weeks, about 3 weeks, about 4 weeks), a certain number of times (e.g., 7, 14, 21, 28, 35, 42, 49, 56, 63, 70, 77, 84, 91, 98, 105, 112, and ranges therebetween) over the span of a protocol period (e.g., an initial period, an intermediate period, a final period), or a certain number of times (e.g., 28, 56, 84, 112, 140, 168, 196, 224, 252, and ranges therebetween) over the span of a protocol. The dosage of the second topical may be adjusted based on the species, concentration, physical form, etc. of the second topical. The second topical may be a Vitamin D analogue or other drug available now or developed in the future.

In some embodiments, phototherapy is administered or received about twice weekly (e.g., substantially twice weekly or exactly twice weekly) while receiving calcitriol. The dosage and/or frequency of the phototherapy may be adjusted based on PASI response, patient tolerance, patient availability, etc. For example, the phototherapy may be administered or received every other day, a certain number of times (e.g., 1, 2, 3, 4, and ranges therebetween) over the span of several days (e.g., about every 1 day, about every 2 days, about every 3 days, about every 4 days, about every 5 days, about every 6 days, about every 7 days, and ranges therebetween), a certain number of times (e.g., 1, 2, 3, 4, and ranges therebetween) over the span of a week, a certain number of times (e.g., 1, 2, 3, 4, 5, 6, 7, 8, 9, 10, 11, 12, 13, 14, 15, 16, 17, 18, 19, 20, 21, 28, 35, 42, 49, 56, 63, 70 and ranges therebetween) over the span of several weeks (e.g., about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 16 weeks, about 20 weeks), a certain number of times (e.g., 1, 2, 3, 4, 5, 6, 7, 8, 9, 10, 11, 12, 13, 14, 15, 16, 17, 18, 19, 20, 21, and ranges therebetween) over the span of a protocol period (e.g., an initial period, an intermediate period, a final period), or a certain number of times (e.g., 1, 2, 3, 4, 5, 6, 7, 8, 9, 10, 11, 12, 13, 14, 15, 16, 17, 18, 19, 20, 21, 28, 35, 42, and ranges therebetween) over the span of a protocol. The protocols herein discuss optional phototherapy administration periods, and it will be appreciated that phototherapy can be administered or received for 1 week, 2 weeks, 3 weeks, 4 weeks, 5 weeks, 6 weeks, 7 weeks, 8 weeks, 9 weeks, 9 weeks, 10 weeks, 11 weeks, 12 weeks, 16 weeks, 20 weeks, and ranges therebetween.

In some embodiments, phototherapy combined with the second topical is received about twice weekly (e.g., substantially twice weekly or exactly twice weekly) for two weeks, for example after the first topical duration (e.g., after four weeks or one month) for a total phototherapy duration of six weeks and twelve doctor visits. During the period in which the second topical is used in conjunction with phototherapy after the period in which the first topical is has been used in conjunction with phototherapy, the phototherapy may cease, for example if a certain PASI is achieved. In some embodiments, phototherapy may be reduced during the period of combination with the second topical, for example to once weekly, three times every two weeks, etc. In general, phototherapy should be maintained for some duration while applying the second topical, but may be ceased (e.g., after about half of the duration of the second topical period, which may be about four weeks or one month). If phototherapy is ceased, the protocol may include a duration when the second topical is the only treatment being applied to the patient.

Treatment using a variety of cellular mechanisms may provide synergy with respect to eliminating the effects of the disease, treating the cause of the disease, and producing normal cellular function. An example of three different cellular mechanisms includes: (1) clobetasol propionate may be used to reduce inflammation, and cells with reduced inflammation may be less prone to proliferate; (2) UV phototherapy may be used to destroy psoriatic lesions and ward off cells displaying psoriatic proliferation; and (3) calcitriol may be used to conduce normal cell proliferation (e.g., by reducing the reproduction rate), differentiation, growth, and maturation.

In embodiments in which the first topical comprises a corticosteroid, for example clobetasol propionate, that was used for four weeks or one month or less, the first topical may be used again after a steroid-free interval. The interval depends on the corticosteroid, but is generally about four weeks or one month. In some embodiments, the period comprises less than about one week, greater than about four weeks, about one week, two weeks, three weeks, four weeks, or ranges therebetween. When use of the first topical is resumed, it may be combined with the second topical. For example, clobetasol propionate and calcitriol may be combined. In some embodiments, the first topical is again received about twice daily (e.g., substantially twice daily or exactly twice daily). The dosage of the first topical may be adjusted based on the species, concentration, physical form, etc. of the first topical and/or based on the second topical. The dosage of the first topical may be the same as or different from the dosage of the first period of treatment (e.g., when combined with phototherapy). In some embodiments, the second topical continues to be received about twice daily (e.g., substantially twice daily or exactly twice daily). The dosage of the second topical may be adjusted based on the species, concentration, physical form, etc. of the second topical and/or based on the first topical. The dosage of the second topical may be the same as or different from the dosage of the second topical in the second period of treatment (e.g., when at least initially combined with phototherapy). In some embodiments, phototherapy may be combined with the first and second topicals. The dosage and/or frequency of the phototherapy may be adjusted based on PASI response, patient tolerance, patient availability, etc. After a period of treatment with the first and second topicals, for example about four weeks or one month, the patient may cease use of all treatments. Psoriasis is not curable, so the patient will experience remission. However, the remission period is expected to be much longer than the remission period of any external agent alone, for example up to six months or even one year.

Topical Vitamin D analogues may begin to lose effectiveness after about four weeks or one month. When used in compliance with certain protocols described herein, for example after combination UV phototherapy and clobetasol propionate, and then in combination with clobetasol propionate, reduced effectiveness of a Vitamin D analogue may be negligible, for example because the psoriasis is already at least PASI 50, at least PASI 75, at least PASI 90, at least PASI 95, or at least PASI 100 and/or because the effects of the clobetasol propionate may mask any reduction in effectiveness.

Compliance with certain protocols described herein can provide numerous advantages over, for example, using any one treatment such as systemic biologics, topicals, or phototherapy. The numerous dangerous side effects of, dependency on, and exorbitant costs of systemic biologics may be avoided. Subject to clinical trials and/or regulatory approvals, compliance with the protocols described herein may be advantageous for children and the elderly. For example, elimination of life-long whole body immunosuppression and toxicity may be particularly beneficial for the elderly, who may already have a weakened immune system, and for children, who would be subject to such the risks of biologics for life. The dangerous side effects of prolonged use of certain topicals such as corticosteroids are also completely avoided. The rate of plaque clearance may be faster, often in only twelve weeks, or as few as six weeks or even as few as four weeks or less. Quick clearance can also enhance patient compliance in response to the positive visual and tangible results. The results after completion of the protocol are better than systemic biologics and any one external agent alone, having better PASI results and/or longer remission periods. After widespread adoption, other advantages will also become apparent.

### THIRD EXAMPLE PROTOCOL

The third example protocol uses a rapidly effective agent first (e.g., combination clobetasol propionate and UV phototherapy), and then transitions to a safer maintenance agent (e.g., combination UV phototherapy and calcitriol and/or calcitriol alone). With this protocol and other protocols described herein, the concerns about lymphoma and other internal cancers, tuberculosis and histoplasmosis, congestive heart failure, demyelinating disorders, etc. are eliminated along with traditional systemic risks such as hepatotoxicity, renal toxicity, and bone marrow suppression. Moreover, instead of spending hours of uncompensated staff time trying to obtain authorization for costly biologic agents, clinicians can be well reimbursed for conducting laser procedures.

The third example protocol includes two distinct periods: Period A and Period B, each of 4 weeks duration. This protocol combines Clobex spray with XTRAC Velocity targeted UV phototherapy as the initial treatment of generalized plaque psoriasis, followed by maintenance phototherapy combined with Vectical ointment.

| Period | Week | Clobex ® | XTRAC ® Velocity | Vectical ® |
|---|---|---|---|---|
| A | 1 | 2/d | 2/wk | |
| | 2 | 2/d | 2/wk | |
| | 3 | 2/d | 2/wk | |
| | 4 | 2/d | 2/wk | |
| B | 5 | | 2/wk | 2/d |
| | 6 | | 2/wk | 2/d |
| | 7 | | | 2/d |
| | 8 | | | 2/d |

### Period A—Weeks 1-4

| Period | Week | Clobex ® | XTRAC ® Velocity | Vectical ® |
|---|---|---|---|---|
| A | 1 | 2/d | 2/wk | |
| | 2 | 2/d | 2/wk | |
| | 3 | 2/d | 2/wk | |
| | 4 | 2/d | 2/wk | |

During Period A (weeks 1 through 4), patients receive Clobex spray about twice daily (e.g., substantially twice daily or exactly twice daily) and patients are administered or receive UV phototherapy treatments about twice weekly (e.g., substantially twice weekly or exactly twice weekly), for example with the XTRAC Velocity. The goal of Period A may be to achieve PASI 75 within four weeks. Other response goals and times are also possible. For example, the goal of Period A may be to achieve PASI 50, PASI 75, PASI 90, PASI 95, or PASI 100, and ranges therebetween, within about 1 week, about 2 weeks, about 4 weeks, and ranges therebetween, and combinations thereof. Administration, application, or receipt of the Clobex spray may be performed by the patient (e.g., at home). For example, receipt of the Clobex spray or other topicals described herein may be as a result of the physician prescribing Clobex spray and the patient applying the Clobex spray. UV phototherapy administration, application, or receipt is currently generally performed at the physician's office since home lasers are generally not available and since patients are generally not trained in laser use.

For the entire 8 weeks of the protocol, laser treatments are preferably at least one day apart (e.g., in some embodiments they do not occur on consecutive days in order to gauge effects of the phototherapy). If the patient misses a laser treatment, the patient can make up that treatment within the week.

In the patient's first visit to the physician, the initial PASI and/or PGA may be calculated. The patient is provided with Clobex spray or a prescription therefor to be used twice daily during Period A. As described herein, a topical corticosteroid other than Clobex may be used, for example comprising clobetasol propionate or another active ingredient, in the form of a spray or other topical such as ointment, emollient, foam, cream, lotion, gel, shampoo, etc. Although the Clobex dosage of the example protocol is twice daily, other dosages may be used depending on the corticosteroid used. For example, a more dilute clobetasol propionate may be used more often or a more concentrated clobetasol propionate may be used less often, subject to such drug approval as may be required in various jurisdictions. Other corticosteroids may have different prescribed treatment dosages.

During the patient's first visit to the physician, the physician may also determine the appropriate laser dosage, for example based on a combination of induration and Fitzpatrick skin phenotype, and administer the initial laser treatment. As described herein, phototherapy other than using the XTRAC Velocity may be used, for example comprising another excimer laser or another UV light source. Although the frequency of the phototherapy is twice weekly, other frequencies may be used depending on, for example, the light source used, the patient's ability to withstand the phototherapy, and/or the patient's response to the phototherapy and/or the Clobex. Additionally during the first visit, the physician may perform a physical exam, ask the patient to fill out a quality of life survey, and take photographs of the patient's front and back.

As the patient already has the Clobex medication, subsequent visits may be limited to XTRAC Velocity treatments and review of progress, side effects, etc. The dosage of each laser treatment may be adjusted as patient tolerance to higher dosages becomes more certain, for example increasing by a small amount each treatment, as otherwise described herein, or based on other criteria. During a week 4 visit, the physician may again ask the patient to fill out a quality of life survey.

### Period B—Weeks 5-8

| Period | Week | Clobex ® | XTRAC ® Velocity | Vectical ® |
|---|---|---|---|---|
| B | 5 | | 2/wk | 2/d |
| | 6 | | 2/wk | 2/d |
| | 7 | | | 2/d |
| | 8 | | | 2/d |

During Period B (weeks 5 through 8), patients receive topical Vectical about twice daily (e.g., substantially twice daily or exactly twice daily). During weeks 5 and 6 of Period B, patients are administered or receive XTRAC Velocity treatments about twice weekly (e.g., substantially twice weekly or exactly twice weekly) for a total of twelve XTRAC Velocity treatments during the protocol. As described herein, the dosages of the phototherapy treatments may be adjusted during each treatment. XTRAC Velocity treatments may also be received in weeks 7 and 8 if needed, for example if a patient achieves a less than PASI 75 response. Patients do not receive Clobex spray during Period B, which is a steroid-free interval of the protocol. The goal of Period B may be to maintain the patient's response using only non-steroid options. As described herein, Vectical may also help the patient to recover from the effects of the Clobex and/or the XTRAC Velocity treatments. Administration, application, or receipt of the Vectical may be performed by the patient (e.g., at home). For example, receipt of the Vectical or other topicals described herein may be as a result of the physician prescribing Vectical and the patient applying the Vectical.

In some embodiments, the third example protocol may be combined with the second example protocol. For example, UV phototherapy may be administered or received about thrice weekly during Period A and not at all during Period B. It will be appreciated that other combinations of the example protocols and other protocols described herein are possible without departing from the spirit of this disclosure.

Week 5 (2 Visits)

In the patient's first visit to the physician in week 5, the PASI and/or PGA may be calculated. The patient is provided with Vectical ointment or a prescription therefor to be used twice daily during Period B, along with clear instructions about ceasing use of Clobex during Period B. As described herein, a topical Vitamin D analogue other than Vectical may be used, for example comprising calcitriol or another active ingredient. Although the Vectical dosage of the example protocol is twice daily, other dosages may be used depending on the Vitamin D analogue used. For example, a more dilute calcitriol may be used more often or a more concentrated calcitriol propionate may be used less often. Other Vitamin D analogues (e.g., Dovonex) may have different prescribed treatment dosages.

The physician also administers an XTRAC Velocity treatment during the first visit in week 5. As described herein, the dosages of the phototherapy treatments may be adjusted during each treatment. Additionally, during the first visit in week 5, the physician may perform a physical exam, confirm adherence to the protocol, ask about side effects, take photographs of the patient's back and front, and reconcile new medications taken by the patient. As the patient already has the Vectical medication, the patient's second visit in week 5 may be limited to XTRAC Velocity treatment and review of progress, side effects, etc. As described herein, the dosages of the phototherapy treatments may be adjusted during each treatment.

Week 6 (2 Visits)

The patient's first visit to the physician in week 6 may be limited to XTRAC Velocity treatment and review of progress, side effects, etc. As described herein, the dosages of the phototherapy treatments may be adjusted during each treatment. In the patient's second visit to the physician in week 6, the PASI and/or PGA may be calculated to determine if the patient will continue XTRAC Velocity treatment past week 6.

Week 7 (0-2 Visits Depending on PASI Response)

If the patient reached PASI 75 response in the last visit, no visits are needed during week 7. In some embodiments in which greater than PASI 75 is desired, phototherapy may continue. In the patient's first visit to the physician in week 7, the PASI and/or PGA may be calculated. Patients with less than PASI 75 response at any point past week 6 may also receive an XTRAC Velocity treatment in that visit or in a subsequent visit. As described herein, the dosages of the phototherapy treatments may be adjusted during each treatment. Additionally during the first visit in week 7, the physician may perform a physical exam, confirm adherence to the protocol, ask about side effects, take photographs of the patient's back and front, and reconcile new medications taken by the patient. An optional second visit in week 7 may be limited to XTRAC Velocity treatment and review of progress, side effects, etc. if needed. As described herein, the dosages of the phototherapy treatments may be adjusted during each treatment.

Week 8 (0-2 Visits Depending on PASI Response)

If the patient had not reached PASI 75 response in the last visit, one or two visits are needed in week 8, each of which may include an XTRAC Velocity treatment, review of progress, side effects, etc. As described herein, the dosages of the phototherapy treatments may be adjusted during each treatment. If the patient reached PASI 75 response in the last visit, only a termination visit may be needed during week 8. In the final or termination visit, the PASI and/or PGA may be calculated and a quality of life survey may be filled out. Additionally during the termination visit, the physician may perform a physical exam, confirm adherence to the protocol, ask about side effects, take photographs of the patient's back and front, reconcile new medications taken by the patient, and complete protocol paperwork.

### FOURTH EXAMPLE PROTOCOL

The fourth example protocol, along with other embodiments disclosed herein, is an entirely new concept for the treatment of generalized, moderate to severe psoriasis that involves a sequential therapy strategy of the “rabbit” to “turtle” transition, e.g., using a rapidly effective agent first (e.g., combination clobetasol propionate and UV phototherapy), and then transitioning to a safer maintenance agent (e.g., combination UV phototherapy and calcitriol and/or calcitriol alone). The fourth example protocol then transitions to a final aggressive maintenance agent (e.g., combination clobetasol propionate and calcitriol). This is a revolutionary concept of treating moderate to severe psoriasis with systemic safety, much greater efficacy than even anti-IL 12/23 agents, and with an attractive reimbursement schedule for the dermatologist (from laser therapy, the billing code for which is already in place). With this protocol, the concerns about lymphoma and other internal cancers, tuberculosis and histoplasmosis, congestive heart failure, demyelinating disorders, etc. are eliminated along with traditional systemic risks such as hepatotoxicity, renal toxicity, and bone marrow suppression. Moreover, instead of spending hours of uncompensated staff time trying to obtain authorization for costly biologic agents, clinicians can be well reimbursed for conducting laser procedures.

This protocol is much more attractive than, for example, simply presenting Vectical as just another “me too” Vitamin D analogue, in which clinicians may question whether Vectical's efficacy is actually non-inferior to Dovonex. In addition, generic calcipotriene may soon come to compete with Vectical. With this protocol, which may revolutionize the treatment of not just localized but also generalized psoriasis, there is likely to be little to no suitable alternative to Vectical, given Dovonex's 100 g/wk limit.

The fourth example protocol includes three distinct periods: Period A, Period B, and Period C, each of 4 weeks duration. This protocol combines Clobex spray with XTRAC Velocity targeted UV phototherapy as the initial treatment of generalized plaque psoriasis, followed by maintenance phototherapy combined with Vectical ointment.

| Period | Week | Clobex ® | XTRAC ® Velocity | Vectical ® |
|---|---|---|---|---|
| A | 1 | 2/d | 2/wk | |
| | 2 | 2/d | 2/wk | |
| | 3 | 2/d | 2/wk | |
| | 4 | 2/d | 2/wk | |
| B | 5 | | 2/wk | 2/d |
| | 6 | | 2/wk | 2/d |
| | 7 | | | 2/d |
| | 8 | | | 2/d |
| C | 9 | 2/d | | 2/d |
| | 10 | 2/d | | 2/d |
| | 11 | 2/d | | 2/d |
| | 12 | 2/d | | 2/d |

### Period A—Weeks 1-4

| Period | Week | Clobex ® | XTRAC ® Velocity | Vectical ® |
|---|---|---|---|---|
| A | 1 | 2/d | 2/wk | |
| | 2 | 2/d | 2/wk | |
| | 3 | 2/d | 2/wk | |
| | 4 | 2/d | 2/wk | |

During Period A (weeks 1 through 4), patients receive Clobex spray about twice daily (e.g., substantially twice daily or exactly twice daily) and patients are administered or receive UV phototherapy treatments about twice weekly (e.g., substantially twice weekly or exactly twice weekly), for example with the XTRAC Velocity. The goal of Period A may be to achieve PASI 75 within four weeks. Other response goals and times are also possible. For example, the goal of Period A may be to achieve PASI 50, PASI 75, PASI 90, PASI 95, or PASI 100, and ranges therebetween, within about 1 week, about 2 weeks, about 4 weeks, and ranges therebetween, and combinations thereof. Administration, application, or receipt of the Clobex spray may be performed by the patient (e.g., at home). For example, receipt of the Clobex spray or other topicals described herein may be as a result of the physician prescribing Clobex spray and the patient applying the Clobex spray. UV phototherapy administration, application, or receipt is currently generally performed at the physician's office since home lasers are generally not available and since patients are generally not trained in laser use.

For the entire 12 weeks of the protocol, laser treatments are preferably at least one day apart (e.g., in some embodiments, they do not occur on consecutive days in order to gauge effects of the phototherapy). If the patient misses a laser treatment, the patient can make up that treatment within the week.

In the patient's first visit to the physician, the initial PASI and/or PGA may be calculated. The patient is provided with Clobex spray or a prescription therefor to be used twice daily during Period A. As described herein, a topical corticosteroid other than Clobex may be used, for example comprising clobetasol propionate or another active ingredient, in the form of a spray or other topical such as ointment, emollient, foam, cream, lotion, gel, shampoo, etc. Although the Clobex dosage of the example protocol is twice daily, other dosages may be used depending on the corticosteroid used. For example, a more dilute clobetasol propionate may be used more often or a more concentrated clobetasol propionate may be used less often, subject to such drug approval as may be required in various jurisdictions. Other corticosteroids may have different prescribed treatment dosages.

During the patient's first visit to the physician, the physician may also determine the appropriate laser dosage, for example based on a combination of induration and Fitzpatrick skin phenotype, and administer the initial laser treatment. All laser treatments should be carried out by personnel trained in the proper use of the XTRAC Velocity. As described herein, phototherapy other than using the XTRAC Velocity may be used, for example comprising another excimer laser or another UV light source. Although the frequency of the phototherapy is twice weekly, other frequencies may be used depending on, for example, the light source used, the patient's ability to withstand the phototherapy, and/or the patient's response to the phototherapy and/or the Clobex. Additionally during the first visit, the physician may perform a physical exam, ask the patient to fill out a quality of life survey, and take photographs of the patient's front and back.

As the patient already has the Clobex medication, subsequent visits may be limited to XTRAC Velocity treatments and review of progress, side effects, etc. The dosage of each laser treatment may be adjusted as patient tolerance to higher dosages becomes more certain, for example increasing by a small amount each treatment, as otherwise described herein, or based on other criteria. During a week 4 visit, the physician may again ask the patient to fill out a quality of life survey.

### Period B—Weeks 5-8

| Period | Week | Clobex ® | XTRAC ® Velocity | Vectical ® |
|---|---|---|---|---|
| B | 5 | | 2/wk | 2/d |
| | 6 | | 2/wk | 2/d |
| | 7 | | | 2/d |
| | 8 | | | 2/d |

During Period B (weeks 5 through 8), patients receive topical Vectical about twice daily (e.g., substantially twice daily or exactly twice daily). During weeks 5 and 6 of Period B, patients are administered or receive XTRAC Velocity treatments about twice weekly (e.g., substantially twice weekly or exactly twice weekly) for a total of twelve XTRAC Velocity treatments during the protocol. XTRAC Velocity treatments may also be administered or received in weeks 7 and 8 if needed, for example if a patient achieves a less than PASI 75 response. Patients do not receive Clobex spray during Period B, which is a steroid-free interval of the protocol. The goal of Period B may be to maintain the patient's response using only non-steroid options. As described herein, Vectical may also help the patient to recover from the effects of the Clobex and/or the XTRAC Velocity treatments. Administration, application, or receipt of the Vectical may be performed by the patient (e.g., at home). For example, receipt of the Vectical or other topicals described herein may be as a result of the physician prescribing Vectical and the patient applying the Vectical.

In some embodiments, the fourth example protocol may be combined with the second example protocol. For example, UV phototherapy may be administered or received about thrice weekly during Period A and not at all during Period B. It will be appreciated that other combinations of the example protocols and other protocols described herein are possible without departing from the spirit of this disclosure.

Week 5 (2 Visits)

In the patient's first visit to the physician in week 5, the PASI and/or PGA may be calculated. The patient is provided with Vectical ointment or a prescription therefor to be used twice daily during Period B, along with clear instructions about ceasing use of Clobex during Period B. As described herein, a topical Vitamin D analogue other than Vectical may be used, for example comprising calcitriol or another active ingredient. Although the Vectical dosage of the example protocol is twice daily, other dosages may be used depending on the Vitamin D analogue used. For example, a more dilute calcitriol may be used more often or a more concentrated calcitriol propionate may be used less often, subject to such drug approval as may be required in various jurisdictions. Other Vitamin D analogues (e.g., Dovonex) may have different prescribed treatment dosages.

The physician also administers an XTRAC Velocity treatment during the first visit in week 5. Additionally during the first visit in week 5, the physician may perform a physical exam, confirm adherence to the protocol, ask about side effects, take photographs of the patient's back and front, and reconcile new medications taken by the patient. As the patient already has the Vectical medication, the patient's second visit in week 5 may be limited to XTRAC Velocity treatment and review of progress, side effects, etc. As described herein, the dosages of the phototherapy treatments may be adjusted during each treatment.

Week 6 (2 Visits)

The patient's first visit to the physician in week 6 may be limited to XTRAC Velocity treatment and review of progress, side effects, etc. As described herein, the dosages of the phototherapy treatments may be adjusted during each treatment. In the patient's second visit to the physician in week 6, the PASI and/or PGA may be calculated to determine if the patient will continue XTRAC Velocity treatment past week 6.

Week 7 (0-2 Visits Depending on PASI Response)

If the patient reached PASI 75 response in the last visit, no visits are needed during week 7. In some embodiments in which greater than PASI 75 is desired, phototherapy may continue. In the patient's first visit to the physician in week 7, the PASI and/or PGA may be calculated. Patients with less than PASI 75 response at any point past week 6 may also receive an XTRAC Velocity treatment in that visit or in a subsequent visit. As described herein, the dosages of the phototherapy treatments may be adjusted during each treatment. Additionally during the first visit in week 7, the physician may perform a physical exam, confirm adherence to the protocol, ask about side effects, take photographs of the patient's back and front, and reconcile new medications taken by the patient. An optional second visit in week 7 may be limited to XTRAC Velocity treatment and review of progress, side effects, etc. if needed. As described herein, the dosages of the phototherapy treatments may be adjusted during each treatment.

Week 8 (0-2 Visits Depending on PASI Response)

If the patient reached PASI 75 response in the last visit, no visits are needed during week 8. If the patient had not reached PASI 75 response in the last visit, one or two visits are needed in week 8, each of which may include an XTRAC Velocity treatment, review of progress, side effects, etc. As described herein, the dosages of the phototherapy treatments may be adjusted during each treatment.

### Period C—Weeks 9-12

| Period | Week | Clobex ® | XTRAC ® Velocity | Vectical ® |
|---|---|---|---|---|
| C | 9 | 2/d | | 2/d |
| | 10 | 2/d | | 2/d |
| | 11 | 2/d | | 2/d |
| | 12 | 2/d | | 2/d |

During Period C (weeks 9 through 12), patients receive Clobex spray about twice daily (e.g., substantially twice daily or exactly twice daily) and patients receive Vectical about twice daily (e.g., substantially twice daily or exactly twice daily). Period C may be a “booster” period in which the goal may be to achieve PASI 90-100.

Week 9 (1-2 Visits Depending on PASI Response)

In the patient's first visit to the physician in week 9, the PASI and/or PGA may be calculated and a quality of life survey may be filled out. The patient is provided with Clobex spray or a prescription therefor to be used twice daily during Period C as well as Vectical for use in Period C if the Period C Vectical was not provided during Period B. In some embodiments, the patient may be provided with the Period C Clobex spray or a prescription therefor in a physician visit during Period A or Period B. However, limiting dispersal of Clobex spray may help with patient compliance of ceasing use during Period B. Patients with less than PASI 75 response in the last visit may also receive an XTRAC Velocity treatment. As described herein, the dosages of the phototherapy treatments may be adjusted during each treatment. Additionally during the first visit in week 9, the physician may perform a physical exam, confirm adherence to the protocol, ask about side effects, take photographs of the patient's back and front, and reconcile new medications taken by the patient.

If the patient reached PASI 75 response in the first visit in week 9, no further visits are needed during week 9. If the patient had not reached PASI 75 response in the first visit in week 9, a second visit in week 9 includes an XTRAC Velocity treatment, review of progress, side effects, etc. As described herein, the dosages of the phototherapy treatments may be adjusted during each treatment.

Weeks 10-12 (1-4 Visits Depending on PASI Response)

If the patient reached PASI 75 response in the last visit in week 9, the only visit in weeks 10-12 is a termination visit in week 12. In the termination visit, the PASI and/or PGA may be calculated and a quality of life survey may be filled out. Additionally during the termination visit, the physician may perform a physical exam, confirm adherence to the protocol, ask about side effects, take photographs of the patient's back and front, reconcile new medications taken by the patient, and complete protocol paperwork.

If the patient had not reached PASI 75 response in the last visit, up to two visits may be needed in each of weeks 10-12, each of which might include an XTRAC Velocity treatment, review of progress, side effects, etc. As described herein, the dosages of the phototherapy treatments may be adjusted during each treatment. The last visit in week 12 is a termination visit as described herein.

### Results

After the twelve weeks of the fourth example protocol, which is much shorter than many previous psoriasis treatment protocols, the rate of plaque clearance is greater than, and the remission period is longer than, for example, using any one treatment such as systemic biologics, topicals, or phototherapy alone. Moreover, the plaques substantially visually clear up quickly, for example after only twelve doctor visits and four or six weeks, thereby providing the patient with visible and tangible results, so patient compliance is very high, which in and of itself is beneficial. In some patients, PASI 95 to PASI 100 has been achieved after four to six weeks.

In comparison to the reported $20,000 to $70,000 yearly cost of some systemic biologics, the fourth example protocol costs about $3,000 to about $4,000 yearly assuming 12 months of remission. Even assuming remission of 6 months, the yearly costs for the fourth example protocol are about $6,000 to about $8,000. If remission is greater than 6 months, costs may be even less.

While synergistic effect was expected, the clinical results using a combination of UV phototherapy and clobetasol propionate are even better than might have been expected. Patients have achieved PASI 95 to PASI 100 after just four to six weeks treatment, the first four of which used a combination of phototherapy and clobetasol propionate and the last two weeks of which used phototherapy alone or in combination with a second topical as described herein.

### FIFTH EXAMPLE PROTOCOL

Certain external treatments are regulatorily limited to treating a certain BSA. For example, Clobex spray is currently only indicated for use up to about 20% BSA. While Clobex spray may in the future be indicated for increased BSA and while other topicals may currently be able to treat greater than 20% BSA, the fifth example protocol is an example of a non-systemic treatment protocol that may be used to treat greater than 20% BSA even if the non-systemic product (e.g., Clobex spray) is regulatorily or otherwise limited to treatment of 20% BSA. The fifth example protocol is also an example of a non-systemic treatment protocol that may be used to treat greater than a certain percentage BSA even if the non-systemic product is regulatorily or otherwise limited to treatment of the certain percentage BSA.

The fifth example protocol includes four or five distinct periods: Period A, Period B, Period C, Period D, and, optionally, Period E, each of 4 weeks duration. This protocol combines Clobex spray with XTRAC Velocity targeted UV phototherapy as the initial treatment of up to 20% BSA psoriasis in two different phases, optionally followed by maintenance phototherapy combined with Vectical ointment. The fifth example protocol is described for a patient with about 40% BSA psoriasis, but the protocol may also be used for greater than about 20% BSA to about 40% BSA or greater than about 40% BSA.

| Period | Week | Clobex ® | XTRAC ® Velocity | Vectical ® |
|---|---|---|---|---|
| A | 1 | 2/d | 2/wk | |
| | 2 | 2/d | 2/wk | |
| | 3 | 2/d | 2/wk | |
| | 4 | 2/d | 2/wk | |
| B | 5 | | 2/wk | 2/d |
| | 6 | | 2/wk | 2/d |
| | 7 | | | 2/d |
| | 8 | | | 2/d |
| C | 9 | 2/d | 2/wk | |
| | 10 | 2/d | 2/wk | |
| | 11 | 2/d | 2/wk | |
| | 12 | 2/d | 2/wk | |
| D | 13 | | 2/wk | 2/d |
| | 14 | | 2/wk | 2/d |
| | 15 | | | 2/d |
| | 16 | | | 2/d |
| (E) | 17 | 2/d | | 2/d |
| | 18 | 2/d | | 2/d |
| | 19 | 2/d | | 2/d |
| | 20 | 2/d | | 2/d |

### Period A—Weeks 1-4

| Period | Week | Clobex ® | XTRAC ® Velocity | Vectical ® |
|---|---|---|---|---|
| A | 1 | 2/d | 2/wk | |
| | 2 | 2/d | 2/wk | |
| | 3 | 2/d | 2/wk | |
| | 4 | 2/d | 2/wk | |

During Period A (weeks 1 through 4), patients receive Clobex spray to a first portion of the psoriasis about twice daily (e.g., substantially twice daily or exactly twice daily) and patients are administered or receive UV phototherapy treatments to the first portion of the psoriasis about twice weekly (e.g., substantially twice weekly or exactly twice weekly), for example with the XTRAC Velocity. Administration, application, or receipt of the Clobex spray may be performed by the patient (e.g., at home). For example, receipt of the Clobex spray or other topicals described herein may be as a result of the physician prescribing Clobex spray and the patient applying Clobex spray. UV phototherapy administration, application, or receipt is currently generally performed at the physician's office since home lasers are generally not available and since patients are generally not trained in laser use. The goal of Period A may be to achieve PASI 75 on the first portion of the psoriasis within four weeks. Other response goals and times are also possible. For example, the goal of Period A may be to achieve PASI 50, PASI 75, PASI 90, PASI 95, or PASI 100, and ranges therebetween, within about 1 week, about 2 weeks, about 4 weeks, and ranges therebetween, and combinations thereof.

In some embodiments, the goal is to eliminate as much plaque as possible as quickly as possible. In certain embodiments in which the patient has less than about 40% BSA (e.g., about 34% BSA), the goal of Period A may be to achieve PASI 50, PASI 75, PASI 90, PASI 95, or PASI 100 on up to about 20% BSA (e.g., with the goal of Period C being to achieve PASI 75 on the remaining about 14% BSA). In embodiments in which the patient has greater than about 40% BSA (e.g., about 48% BSA), the goal of Period A may be to achieve PASI 50, PASI 75, PASI 90, PASI 95, or PASI 100 on up to about 20% BSA (e.g., with the goal of Period C being to achieve PASI 50, PASI 75, PASI 90, PASI 95, or PASI 100 on about 20% BSA and the goal of an additional Clobex and phototherapy period to achieve PASI 50, PASI 75, PASI 90, PASI 95, or PASI 100 on the remaining about 8% BSA.

In some embodiments, the goal is to eliminate a fraction of the plaque during each period. In certain embodiments in which the patient has less than about 40% BSA (e.g., about 34% BSA), the goal of Period A may be to achieve PASI 50, PASI 75, PASI 90, PASI 95, or PASI 100 on half of the psoriasis (e.g., about 17% BSA, with the goal of Period C being to achieve PASI 50, PASI 75, PASI 90, PASI 95, or PASI 100 on the remaining about 17% BSA). In embodiments in which the patient has greater than about 40% BSA (e.g., about 48% BSA), the goal of Period A may be to achieve PASI 50, PASI 75, PASI 90, PASI 95, or PASI 100 on a fraction of the psoriasis (e.g., about 16% BSA, with the goal of Period C being to achieve PASI 50, PASI 75, PASI 90, PASI 95, or PASI 100 on about 16% BSA and the goal of an additional Clobex and phototherapy period to achieve PASI 50, PASI 75, PASI 90, PASI 95, or PASI 100 on the remaining about 16% BSA; about 16% BSA).

For the entire 16 or 20 weeks of the protocol, laser treatments are preferably at least one day apart (e.g., in some embodiments they do not occur on consecutive days in order to gauge effects of the phototherapy). If the patient misses a laser treatment, the patient can make up that treatment within the week.

In the patient's first visit to the physician, the initial PASI and/or PGA may be calculated. The patient is provided with Clobex spray or a prescription therefor to be used twice daily during Period A. As described herein, a topical corticosteroid other than Clobex may be used, for example comprising clobetasol propionate or another active ingredient, in the form of a spray or other topical such as ointment, emollient, foam, cream, lotion, gel, shampoo, etc. Although the Clobex dosage of the example protocol is twice daily, other dosages may be used depending on the corticosteroid used. For example, a more dilute clobetasol propionate may be used more often or a more concentrated clobetasol propionate may be used less often, subject to such drug approval as may be required in various jurisdictions. Other corticosteroids may have different prescribed treatment dosages.

During the patient's first visit to the physician, the physician may also determine the appropriate laser dosage, for example based on a combination of induration and Fitzpatrick skin phenotype, and administer the initial laser treatment. All laser treatments should be carried out by personnel trained in the proper use of the XTRAC Velocity. As described herein, phototherapy other than using the XTRAC Velocity may be used, for example comprising another excimer laser or another UV light source. Although the frequency of the phototherapy is twice weekly, other frequencies may be used depending on, for example, the light source used, the patient's ability to withstand the phototherapy, and/or the patient's response to the phototherapy and/or the Clobex. Additionally during the first visit, the physician may perform a physical exam, ask the patient to fill out a quality of life survey, and take photographs of the patient's front and back.

As the patient already has the Clobex medication, subsequent visits may be limited to XTRAC Velocity treatments of the first portion and review of progress, side effects, etc. The dosage of each laser treatment may be adjusted as patient tolerance to higher dosages becomes more certain, for example increasing by a small amount each treatment, as otherwise described herein, or based on other criteria. During a week 4 visit, the physician may again ask the patient to fill out a quality of life survey.

In some embodiments, phototherapy, which is not limited to 20% BSA, may be used on portions other than the first portion, for example to get a jump start on treatment of the other portion(s).

### Period B—Weeks 5-8

| Period | Week | Clobex ® | XTRAC ® Velocity | Vectical ® |
|---|---|---|---|---|
| B | 5 | | 2/wk | 2/d |
| | 6 | | 2/wk | 2/d |
| | 7 | | | 2/d |
| | 8 | | | 2/d |

During Period B (weeks 5 through 8), patients receive topical Vectical to the first portion about twice daily (e.g., substantially twice daily or exactly twice daily). Vectical is currently indicated for use up to 35% BSA, so patients may be administered Vectical to portions of the psoriasis other than the first portion, as well. During weeks 5 and 6 of Period B, patients are administered or receive XTRAC Velocity treatments to the first portion about twice weekly (e.g., substantially twice weekly or exactly twice weekly) for a total of twelve XTRAC Velocity treatments on the first portion during the protocol. XTRAC Velocity treatments may also be administered or received in weeks 7 and 8 if needed, for example if a patient achieves a less than PASI 75 response on the first portion. Patients do not receive Clobex spray during Period B, which is a steroid-free interval of the protocol. The goal of Period B may be to maintain the patient's response in the first portion using only non-steroid options. As described herein, Vectical may also help the patient to recover from the effects of the Clobex and/or the XTRAC Velocity treatments. Administration, application, or receipt of the Vectical may be performed by the patient (e.g., at home). For example, receipt of the Vectical or other topicals described herein may be as a result of the physician prescribing Vectical and the patient applying the Vectical.

In some embodiments, the fifth example protocol may be combined with the second example protocol. For example, UV phototherapy may be administered or received about thrice weekly to the first portion during Period A and not at all during Period B. It will be appreciated that other combinations of the example protocols and other protocols described herein are possible without departing from the spirit of this disclosure.

Week 5 (2 Visits)

In the patient's first visit to the physician in week 5, the PASI and/or PGA may be calculated. The patient is provided with Vectical ointment or a prescription therefor to be used twice daily on at least the first portion during Period B, along with clear instructions about ceasing use of Clobex during Period B. As described herein, a topical Vitamin D analogue other than Vectical may be used, for example comprising calcitriol or another active ingredient. Although the Vectical dosage of the example protocol is twice daily, other dosages may be used depending on the Vitamin D analogue used. For example, a more dilute calcitriol may be used more often or a more concentrated calcitriol propionate may be used less often, subject to such drug approval as may be required in various jurisdictions. Other Vitamin D analogues (e.g., Dovonex) may have different prescribed treatment dosages.

The physician also administers an XTRAC Velocity treatment on the first portion during the first visit in week 5. Additionally during the first visit in week 5, the physician may perform a physical exam, confirm adherence to the protocol, ask about side effects, take photographs of the patient's back and front, and reconcile new medications taken by the patient. As the patient already has the Vectical medication, the patient's second visit in week 5 may be limited to XTRAC Velocity treatment and review of progress, side effects, etc. As described herein, the dosages of the phototherapy treatments may be adjusted during each treatment.

Week 6 (2 Visits)

The patient's first visit to the physician in week 6 may be limited to XTRAC Velocity treatment on the first portion and review of progress, side effects, etc. As described herein, the dosages of the phototherapy treatments may be adjusted during each treatment. In the patient's second visit to the physician in week 6, the PASI and/or PGA may be calculated to determine if the patient will continue XTRAC Velocity treatment on the first portion past week 6.

Week 7 (0-2 Visits Depending on PASI Response)

If the patient reached PASI 75 response in the first portion in the last visit, no visits are needed during week 7. In some embodiments in which greater than PASI 75 is desired in the first portion (e.g., for additional clearance before treating portions other than the first portion), phototherapy on the first portion may continue. In the patient's first visit to the physician in week 7, the PASI and/or PGA may be calculated. Patients with less than PASI 75 response at any point past week 6 may also receive an XTRAC Velocity treatment on the first portion in that visit or in a subsequent visit. As described herein, the dosages of the phototherapy treatments may be adjusted during each treatment. Additionally during the first visit in week 7, the physician may perform a physical exam, confirm adherence to the protocol, ask about side effects, take photographs of the patient's back and front, and reconcile new medications taken by the patient. An optional second visit in week 7 may be limited to XTRAC Velocity treatment on the first portion and review of progress, side effects, etc. if needed. As described herein, the dosages of the phototherapy treatments may be adjusted during each treatment.

Week 8 (0-2 Visits Depending on PASI Response)

If the patient reached PASI 75 response in the first portion in the last visit, no visits are needed during week 8. If the patient had not reached PASI 75 response in the last visit, one or two visits are needed in week 8, each of which may include an XTRAC Velocity treatment on the first portion, review of progress, side effects, etc. As described herein, the dosages of the phototherapy treatments may be adjusted during each treatment.

### Period C—Weeks 9-12

| Period | Week | Clobex ® | XTRAC ® Velocity | Vectical ® |
|---|---|---|---|---|
| C | 9 | 2/d | 2/wk | |
| | 10 | 2/d | 2/wk | |
| | 11 | 2/d | 2/wk | |
| | 12 | 2/d | 2/wk | |

During Period C (weeks 9 through 12), patients receive Clobex spray to a second portion of the psoriasis about twice daily (e.g., substantially twice daily or exactly twice daily) and patients are administered or receive UV phototherapy treatments to the second portion of the psoriasis about twice weekly (e.g., substantially twice weekly or exactly twice weekly), for example with the XTRAC Velocity. The goal of Period C may be to achieve PASI 50, PASI 75, PASI 90, PASI 95, or PASI 100 on the second portion of the psoriasis within four weeks. Other than treating the second portion rather than the first portion, Period C may be substantially identical to Period A.

In some embodiments in which the patient has about 40% BSA or less, the second portion comprises the initial BSA minus the first portion. For example, if the initial BSA was about 40% and the first portion was about 20% BSA, the second portion comprises about 20%. For another example, if the initial BSA was about 34% and the first portion was about 20% BSA, the second portion comprises about 14%. For yet another example, if the initial BSA was about 34% and the first portion was about 17% BSA, the second portion comprises about 17%.

In some embodiments in which the patient has greater than about 40% BSA, the second portion comprises the initial BSA minus the first portion minus a third portion to be treated later. For example, if the initial BSA was about 48% and the first portion was about 20% BSA, the second portion may comprise about 20%, the third portion comprising about 8%. For another example, if the initial BSA was about 48% and the first portion was about 16% BSA, the second portion comprises about 16%, the third portion comprising about 16%. For yet another example, if the initial BSA was about 48% and the first portion was about 20% BSA, the second portion comprises about 14%, the third portion comprising about 14%.

In some embodiments, phototherapy, which is not limited to 20% BSA, may be used on portions other than the second portion, for example to further treat stubborn plaques in the first portion and/or to get a jump start on treatment of the other portion(s).

### Period D—Weeks 13-16

| Period | Week | Clobex ® | XTRAC ® Velocity | Vectical ® |
|---|---|---|---|---|
| D | 13 | | 2/wk | 2/d |
| | 14 | | 2/wk | 2/d |
| | 15 | | | 2/d |
| | 16 | | | 2/d |

During Period D (weeks 13 through 16), patients receive topical Vectical to the second portion about twice daily (e.g., substantially twice daily or exactly twice daily). Vectical is currently indicated for use up to 35% BSA, so patients may receive Vectical to portions of the psoriasis other than the second portion, as well. During weeks 13 and 14 of Period D, patients are administered or receive XTRAC Velocity treatments to the second portion about twice weekly (e.g., substantially twice weekly or exactly twice weekly) for a total of twelve XTRAC Velocity treatments on the second portion during the protocol. XTRAC Velocity treatments may also be administered or received in weeks 15 and 16 if needed, for example if a patient achieves a less than PASI 75 response on the second portion. Patients do not receive Clobex spray during Period D, which is another steroid-free interval of the protocol. The goal of Period D may be to maintain the patient's response in the second portion using only non-steroid options. As described herein, Vectical may also help the patient to recover from the effects of the Clobex and/or the XTRAC Velocity treatments. Other than treating the second portion rather than the first portion, Period D may be substantially identical to Period B. In embodiments in which the patient has greater than about 40% BSA, additional periods substantially identical to Periods A and B and/or Periods C and D may be performed to treat the remaining portion(s).

### Period E—Weeks 9-12

| Period | Week | Clobex ® | XTRAC ® Velocity | Vectical ® |
|---|---|---|---|---|
| E | 17 | 2/d | | 2/d |
| | 18 | 2/d | | 2/d |
| | 19 | 2/d | | 2/d |
| | 20 | 2/d | | 2/d |

During optional Period E (weeks 17 through 20), patients receive Clobex spray about twice daily (e.g., substantially twice daily or exactly twice daily) and patients receive Vectical about twice daily (e.g., substantially twice daily or exactly twice daily). Administration may be to areas that still appear affected.

Week 17 (1-2 Visits Depending on PASI Response)

In the patient's first visit to the physician in week 17, the PASI and/or PGA may be calculated and a quality of life survey may be filled out. The patient is provided with Clobex spray or a prescription therefor to be used twice daily during Period E as well as Vectical for use in Period E if the Period E Vectical was not provided during Period B or Period D. In some embodiments, the patient may be provided with the Period E Clobex spray or a prescription therefor in a physician visit during Period A through Period D. However, limiting dispersal of Clobex spray may help with patient compliance of ceasing use during Period B and Period D. Patients with less than PASI 75 response on any portion of the psoriasis may also receive an XTRAC Velocity treatment. As described herein, the dosages of the phototherapy treatments may be adjusted during each treatment. Additionally during the first visit in week 17, the physician may perform a physical exam, confirm adherence to the protocol, ask about side effects, take photographs of the patient's back and front, and reconcile new medications taken by the patient.

If the patient reached PASI 75 response on all portions of the psoriasis in the first visit in week 17, no further visits are needed during week 17. If the patient had not reached PASI 75 response in any portion of the psoriasis in the first visit in week 17, a second visit in week 17 includes an XTRAC Velocity treatment, review of progress, side effects, etc. As described herein, the dosages of the phototherapy treatments may be adjusted during each treatment.

Weeks 18-20 (1-4 Visits Depending on PASI Response)

If the patient reached PASI 75 response on all portions of the psoriasis in the last visit in week 17, the only visit in weeks 18-20 is a termination visit in week 20. In the termination visit, the PASI and/or PGA may be calculated and a quality of life survey may be filled out. Additionally during the termination visit, the physician may perform a physical exam, confirm adherence to the protocol, ask about side effects, take photographs of the patient's back and front, reconcile new medications taken by the patient, and complete protocol paperwork.

If the patient had not reached PASI 75 response on all portions of the psoriasis in the last visit, up to two visits may be needed in each of weeks 18-20, each of which might include an XTRAC Velocity treatment, review of progress, side effects, etc. As described herein, the dosages of the phototherapy treatments may be adjusted during each treatment. The last visit in week 20 is a termination visit as described herein.

### Clinical Trial Results

An ongoing clinical trial (NCT01012713) is being performed by the inventors in accordance with the protocols described herein. Inclusion criteria include (1) male or female subjects 18 years of age of older; (2) subjects should have ≧10%, but not more than 20% total body involvement of stable plaque type psoriasis; (3) subjects must have BMI<30 and weigh less than 250 pounds; (4) subjects must have Fitzpatrick Skin Type II or above; (5) subjects must be able to discontinue any topical therapy (other than emollients) or received UVB phototherapy two weeks prior to starting the study; (6) subjects must be able to discontinue any biologic or systemic agents or PUVA photochemotherapy four weeks prior to starting the study; (7) subject is able to complete the study and to comply with study instructions; (8) subject is capable of understanding and willing to provide signed and dated written voluntary informed consent (and any local or national authorization requirements) before any protocol specific procedures are performed; and (9) any additional diagnoses must, in the investigator's opinion, not preclude the subject from safely participating in this study or interfere with the evaluation of the subject's psoriasis. Exclusion criteria include (1) subject is younger than 18 years of age; (2) subject has less than 10% or greater than 20% body surface involvement of his/her psoriasis; (3) history of known or suspected intolerance to any of the ingredients of the investigational study product; (4) subject has a photosensitivity disorder (such as lupus, etc.) or a history of clinically significant photosensitivity; (5) subjects possess other diagnoses that, in the investigator's opinion, preclude him/her from safely participating in this study or interfere with the evaluation of the subject's psoriasis; (6) subject is not willing to discontinue topical treatment (other than emollients) or UVB phototherapy for two weeks prior to starting the study; (7) subject is not willing to discontinue biologic or systemic agents or PUVA photochemotherapy for four weeks prior to starting the study; (8) subject has psoriatic involvement only on the hands, feet, or scalp; (9) subject has been diagnosed with unstable or non-plaque forms of psoriasis, including guttate, erythrodermic, exfoliative, or pustular psoriasis; (10) subject has a history of koebnerization phenomenon; (11) subject has keloids or past history of keloid formation; (12) subject has melanoma or past history of melanoma; (13) subject has active (cutaneous) invasive non-melanoma skin cancer (NMSC); (14) subject is determined not be a candidate for phototherapy by the investigator; (15) subject has used other investigational drugs within four weeks prior to the study; and (16) subject is known, or suspected of being unable to comply with the study protocol. The specific protocol being studied involves the following treatment periods:

| Period | Week | Clobex ® | XTRAC ® Velocity | Vectical ® |
|---|---|---|---|---|
| A | 1 | 2/d | 2/wk | |
| | 2 | 2/d | 2/wk | |
| | 3 | 2/d | 2/wk | |
| | 4 | 2/d | 2/wk | |
| B | 5 | | 2/wk | 2/d |
| | 6 | | 2/wk | 2/d |
| | 7 | | as needed | 2/d |
| | 8 | | as needed | 2/d |
| C | 9 | 2/d | as needed | |
| | 10 | 2/d | as needed | |
| | 11 | 2/d | as needed | |
| | 12 | 2/d | as needed | |

The Clobex® is a spray having a concentration of 0.05% clobetasol propionate, and is applied by the subject BID (bis in die, or two times per day) at home. The phototherapy is performed by a clinician using an XTRAC® Velocity 700 excimer laser having a dosage between 300 mJ/cm² and 1091 mJ/cm², which is adjusted throughout treatment based on patient response as described herein. The Vectical® is an ointment having a concentration of 3 μg/g calcitriol, and is applied by the subject BID at home.

Fourteen subjects have been enrolled in the clinical trial, of whom twelve have completed six weeks the protocol and two subjects have ongoing treatment. Ten of the twelve subjects (about 83%) who completed the protocol achieved PASI 75 after only six weeks of treatment. These clearance results are unexpectedly better than the three leading systemic biologics after twelve weeks (about 34% PASI 75 for Enbrel®, about 68% PASI 75 for Humira®, and about 68% PASI 75 for Stelara®). That is, the preliminary results show better clearance in half the treatment time. Eight subjects completed all twelve weeks of the protocol, and seven of those eight subjects (about 88%) achieved PASI 75. Thus, the preliminary results show much better clearance (almost 30% improvement over the most effective systemic biologic) in the same period of time.

Three of the twelve subjects who completed the protocol have been reviewed after completion of the protocol. Two of the three subjects had no remission after six months, and the third subject had no remission after one year. This 0% remission result is unexpectedly better than, for example, laser therapy alone, which generally has about 80% remission after six months for subjects starting with 10%-30% BSA psoriasis.

The effectiveness of the protocol in such a short duration is unexpected for a variety of reasons. For example, there are generally two schools of thought with respect to effective psoriasis treatment: (1) use systemic biologics, which are fast but dangerous; or (2) use external phototherapy or topicals, which are slow but safe. The first school of thought, systemic biologics, were thought by skilled artisans to have superior results to external treatment, so systemic immunosuppression and the resulting risk of infection and cancer was accepted. However, the inventors have demonstrated that external treatment according to the protocols described herein is safe and effective, as well as fast. Indeed, the results are unexpectedly more effective and faster than any of the previous treatments. Some subjects have experienced expected side effects such as mild skin irritation and mild erythema. However, there have been no unexpected side effects, for example due to the previously unexplored interaction between phototherapy and topicals.

Although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. For example, the dosages of the non-systemic treatments described herein may be adjusted based on regulatory approval, modifications to existing products, development of new products, etc. For example, clobetasol propionate concentrations less than about 0.05% (e.g., about 0.01%, about 0.02%, about 0.025%, about 0.03%, about 0.04%) and/or greater than about 0.05% (e.g., about 0.06%, about 0.07%, about 0.075%, about 0.08%, about 0.09%, about 0.1%, about 0.15%), calcitriol concentrations less than about 3 μg/g (e.g., about 0.5 μg/g, about 1 μg/g, about 1.5 μg/g, about 2 μg/g, about 2.5 μg/g) and/or greater than about 3 μg/g, (e.g., about 3.5 μg/g, about 4 μg/g, about 4.5 μg/g, about 5 μg/g, about 5.5 μg/g, about 6 μg/g, about 9 μg/g), and dosages or formulations of other non-systemic treatments now available or developed in the future (including phototherapy devices) may be used with the protocols described herein. For another example, the variables of the protocols described herein (e.g., dosages, frequencies, periods, treatment goals, etc.) may be adjusted based on values provided herein or otherwise. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Thus, it is intended that the scope of the invention herein disclosed should not be limited by the particular embodiments described above. Additionally, due to possible of non-compliance (e.g., accidental) with one of the aspects of the protocols described herein, it will be appreciated that substantially following a protocol may be considered equivalent to following a protocol under certain circumstances.

1.-27. (canceled)

28. A method of treating a skin condition, the method comprising:
administering UV phototherapy about twice per week during a first period in which clobetasol propionate spray is applied about twice per day; and
administering UV phototherapy about twice per week during a second period in which calcitriol ointment is applied about twice per day.

29. The method of claim 28, wherein the skin condition comprises psoriasis.

30. The method of claim 28, wherein the first period is about 4 weeks or about 1 month.

31.-35. (canceled)

36. A method of treating a skin condition, the method comprising:
administering UV phototherapy during a first period in which a topical corticosteroid is received; and
administering UV phototherapy during a second period in which a topical Vitamin D analogue is received.

37. The method of claim 36, wherein the skin condition comprises psoriasis.

38. The method of claim 36, wherein the first period is about 4 weeks or about 1 month.

39. The method of claim 36, wherein the topical corticosteroid comprises clobetasol propionate.

40. The method of claim 36, wherein the Vitamin D analogue comprises calcitriol.

41.-55. (canceled)

56. A method of treating a skin condition, the method comprising administering UV phototherapy during a period in which a topical corticosteroid is received, wherein after the period, response of the psoriasis is better than response to UV phototherapy alone or clobetasol propionate alone.

57. The method of claim 56, wherein the skin condition comprises psoriasis.

58. The method of claim 56, wherein the period is about 4 weeks or about 1 month.

59. The method of claim 56, wherein the topical corticosteroid comprises clobetasol propionate.

60. A method of treating a skin condition, the method comprising using a combination of at least two non-systemic treatments, wherein, after a treatment duration of about 12 weeks or less, the skin condition has improved by at least about 75%.

61. The method of claim 60, wherein the skin condition comprises psoriasis.

62. The method of claim 60, wherein the at least two non-systemic treatments include UV phototherapy and a non-systemic treatment selected from the group consisting of a topical corticosteroid and a Vitamin D analogue.

63. The method of claim 62, wherein the topical corticosteroid comprises clobetasol propionate.

64. The method of claim 62, wherein the Vitamin D analogue comprises calcitriol.

65. The method of claim 60, wherein the treatment duration is about 6 weeks or less.

66. (canceled)

67. The method of claim 28, wherein the second period is about 2 weeks.

68. The method of claim 28, wherein the second period is contiguous with the first period.

69. The method of claim 36, wherein the second period is about 2 weeks.

70. The method of claim 36, wherein the second period is contiguous with the first period.
